(19) United States
(12) Patent Application Publication
(10) Pub. No.: US 2007/0166336 A1
(43) Pub. Date: Jul. 19, 2007

(54) Stable and palatable oral liquid sumatriptan compositions

(76) Inventors: David Delmarre, Vernon Hills, IL (US); Ajit S. Narang, Gurnee, IL (US); Kaidi Zhao, Evanston, IL (US); Meagan E. Weldele, Overland Park, KS (US); Barbara Chen, Northbrook, IL (US); Carlos-Julian Centeno, Chicago, IL (US); Danchen Gao, Chicago, IL (US)

Correspondence Address: WINSTON & STRAWN LLP;PATENT DEPARTMENT, 1700 K STREET, N.W., WASHINGTON, DC, 20006, US

(21) Appl. No.: 11/637,677

(22) Filed: Dec. 13, 2006

Related U.S. Application Data
(60) Provisional application No. 60/749,585, filed on Dec. 13, 2005.

(51) Int. Cl. A61K 31/405; A61K 9/00

(52) U.S. Cl. 424/400; 514/419

(57) ABSTRACT

The present invention is directed to improved oral liquid compositions that include sumatriptan, or a pharmaceutically acceptable salt or metabolite thereof, and a pharmaceutically acceptable carrier that includes a liquid portion of the composition. The compositions are substantially free of oxidation impurities. Typically, the compositions include a sweetening agent and a flavoring agent, or a bitterness-reducing agent and flavoring agent. Processes of preparing such compositions and methods of administering such compositions are also included.

### CROSS-REFERENCES TO RELATED APPLICATIONS

This application claims the benefit of provisional application No. 60/749,585, filed Dec. 13, 2005, the entire contents of which is incorporated herein by express reference thereto.

### TECHNICAL FIELD

The present invention relates generally to oral liquid sumatriptan compositions and processes for their preparation. The invention further relates to methods of treatment by administering the compositions.

### BACKGROUND OF THE INVENTION

Sumatriptan is an agonist for a vascular 5-HT₁ receptor subtype, a member of the 5-HT_{1D} family. The vascular 5-HT₁ receptor subtype that sumatriptan activates is present on the human basilar artery, and in the vasculature of human aura mater and mediates vasoconstriction. This action in humans correlates with the relief of migraine headaches. Sumatriptan succinate is indicated for the acute treatment of migraine attacks with or without aura in adults.

Suppression of migraine headaches is thought to result from sumatriptan-induced decreases in the firing of serotonergic (5-hydroxytryptaminergic, 5-HT) neurons. Specifically, it is thought that agonist activity at the 5-HT_{1D} receptor subtype provides relief of acute headache. Sumatriptan is a highly selective agonist of this receptor subtype and has no significant activity at other 5-HT receptor subtypes or at adrenergic, dopaminergic, muscarinic, or benzodiazepine receptors.

It has been proposed that constriction of cerebral blood vessels resulting from 5-HT_{1D} receptor stimulation reduces the pulsation that may be responsible for the pain of vascular headaches. Studies in humans have shown that blood flow velocity in the middle cerebral arteries is significantly reduced during a migraine on the side of the headache, and that relief is accompanied by return of the blood flow velocity in these vessels to normal. Other studies, however, have not consistently shown a significant correlation between dilatation of cerebral blood vessels and pain or other symptoms of migraine headaches, or between medication-induced vasoconstriction and relief of these headaches. Sumatriptan may also relieve migraines by decreasing release of neuropeptides and other mediators of inflammation and by reducing extravasations of plasma proteins.

Sumatriptan succinate, a pharmaceutically acceptable salt of sumatriptan, is a white to off-white free flowing powder with a molecular weight of 413.5. It is readily soluble in water and in saline. Sumatriptan succinate is also known as 3-[2-(dimethylamino)ethyl]-N-methyl-indole-5-methanesulfonamide succinate (1:1). Its empirical formula is C₁₄H₂₁N₃O₂S.C₄H₆O₄ and it has the following structural formula: 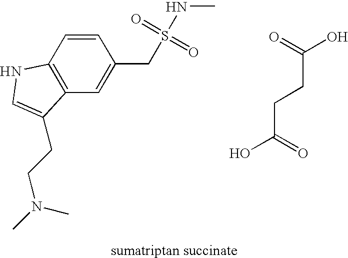
There are three important pKa values for sumatriptan succinate: pKa₁ (succinic acid) 4.21 and 5.67, pKa₂ (tertiary amine group) 9.63, and pKa₃ (sulfonamide group) >12.

Sumatriptan is rapidly but incompletely absorbed following oral administration and undergoes first-pass metabolism resulting in a low absolute bioavailability of about 14%. The 20 mg nasal spray presently available also has relatively low bioavailability of 17% compared to 97% bioavailability for the 6 mg subcutaneous injection. The Tₘₐₓ for a 50 mg tablet is 2.5 hours, while the spray is 1 hour, and the subcutaneous injection is 0.2 hour. Plasma protein binding is low at about 14 to 21%. The elimination half-life of sumatriptan is about 2 hours and it is extensively metabolized in the liver by monoamine oxidase A. Treatment with monoamine oxidase inhibitors (MAOIs) such as moclobemide generally leads to an increase of sumatriptan plasma levels and it is not advisable to co-administer the compounds. Sumatriptan is excreted mainly in the urine as the inactive indole acetic acid derivative and its glucuronide.

Sumatriptan succinate may be used alone or in combination with other pharmaceutical agents. The usual dose of sumatriptan for the treatment of migraine ranges from 0.1 mg to 100 mg, for example, from 0.5 mg to 50 mg, but usually 2 mg to 40 mg per dose in solid tablets that are commercially available. The doses can be administered up to eight times per day, but usually one to four times per day, not to exceed a total daily dose of 200 mg.

A wide variety of active pharmaceutical agents, including sumatriptan, exhibit the undesirable characteristic of bitter taste production either during or immediately after oral administration. Any bitter flavor of a pharmaceutical agent in a liquid formulation is typically detected during the drinking process or immediately after swallowing. Additionally, the bitter flavor of a pharmaceutical agent in a tablet, capsule, suspension or other oral dosage form is typically readily detected upon administration if the bitter agent is brought into contact with the taste buds, e.g., as by sustained holding of the dosage form in the mouth, by inadvertent chewing of the dosage form, or by some other release of the bitter pharmaceutical agent in the mouth.

The administration of an oral dosage form is generally the preferred route of administration of many pharmaceutical agents because it provides for easy, low-cost administration. Patient compliance, however, can sometimes be a factor when a patient is requested to swallow a bitter tablet, capsule or liquid formulation. Patients give many reasons for their refusal or inability to accept the oral administration of a medicinal such as unattractive presentation, overlarge size, bad taste or simple fear that an unchewed dosage form may catch in the throat. Patients who have difficulties with oral dosage forms, particularly solid oral dosage forms, often exhibit a gag reflex which effectively prevents oral administration. This problem is common in, but not specific to, children.

Sumatriptan and its physiologically acceptable salts have an unpleasant bitter taste profile and, when administered orally, may undesirably intensify the nausea and vomiting associated with migraines. This limits the use of sumatriptan orally, which is considered to be the most widely accepted and convenient route of administration. Successful masking of the unpleasant taste is a key element for patients' acceptance and compliance of an oral dosage form.

Various techniques to mask the unpleasant taste of sumatriptan have been tried. When the medicament is solid, such as tablets or granules, a bitter taste or the like can be masked relatively easily by coating or incorporation of the medicament in a matrix that masks the bitter taste. For example, U.S. Pat. Nos. 5,863,559; 6,020,001; and 6,368,627 each note the unpleasant taste of sumatriptan, and report that the unpleasant taste associated with oral administration of sumatriptan is substantially eliminated by the use of sumatriptan film-coated tablet dosage forms. Specifically, the '559 patent, the '001 patent, and the '627 patent each disclose a film coated tablet that includes a tablet core containing sumatriptan or a pharmaceutically acceptable salt or solvate thereof as the active ingredient. The core is substantially covered with a coating that includes a film forming polymer, such as hydroxypropylmethylcellulose, hydroxypropylcellulose or methylcellulose, and copolymers of methacrylic acid and methyl methacrylate polymers.

Similarly, International Application No. WO 01/37816 discloses a process for the coating of sumatriptan tablet cores and tablets to reportedly provide taste masking of the sumatriptan. The process includes spraying a coating solution or suspension of a sugar, a starch, or a mixture of a sugar and a starch, onto tablet cores to obtain coated tablets.

Other methods of masking the taste of sumatriptan in solid dosage forms have been reported. For example, International Publication No. WO 02/41920 teaches blending active ingredients, including sumatriptan, with cyclodextrin to mask the taste of the active. International Publication No. WO 2004/009085 teaches uncoated taste-masked sumatriptan tablets and processes for their preparation. The '085 publication reports that taste-masking properties can be imparted to the solid dosage form by granulating sumatriptan with one or more diluents and/or binders, mixing the granulated sumatriptan granules with other pharmaceutically excipients, and compressing to form a tablet. Granulation with one or more diluents and/or binders allegedly provides a uniform or substantially uniform layer or encapsulation over or around the individual sumatriptan particles and thereby is reported to mask the unpleasant taste associated with sumatriptan.

In addition, International Publication No. WO 2004/084865 and related U.S. Publication No. 2004/0191298 each disclose various formulations that include one or more pharmaceutically active ingredients, including sumatriptan, and cocoa powder as a taste masker, filler, and texturizer. Generally, the formulation has a weight of around 200 mg to 1000 mg and includes one or more pharmaceutically active ingredients, one or more lipid ingredients, cocoa powder, water-soluble or dispersible diluents, one or more sweetening agents, one or more buffering agents, one or more flavoring agents, one or more taste modifiers, one or more emulsifiers/solubilisers, and one or more coloring agents.

For liquids, it is the common practice to alleviate the bitter taste of pharmaceuticals with sweetening and flavoring agents. U.S. Pat. No. 5,037,845 describes and claims sumatriptan and its physiologically acceptable salts, including the succinate. The '845 patent describes sumatriptan as useful in treating and/or preventing pain resulting from dilatation of the cranial vasculature, in particular migraine and related disorders such as cluster headache. The '845 patent alleges that liquid preparations for oral administration may take essentially any form, such as aqueous or oily solutions, syrups, elixirs, emulsions or suspensions, or they may be presented as a dry product for constitution with water or other suitable vehicle before use. The liquid preparations of the '845 patent are allegedly prepared by conventional means with pharmaceutically acceptable additives such as suspending agents, emulsifying agents, non-aqueous vehicles, and preservatives. The liquid preparations may also contain conventional buffers, flavoring, coloring, and sweetening agents as appropriate. One particular formulation of a sumatriptan syrup included hydroxypropylmethylcellulose, buffer, flavor, color, preservative, sweetener, and purified water, while the sumatriptan suspension was stated to include aluminum monostearate, sweetening agent, flavor, color, and fractionated coconut oil.

U.S. Pat. No. 5,744,482 and related references European Patent Application No. EP 0 747 049, and International Publication Nos. WO 96/24353 and WO 96/29074 each disclose a suspension formulation that include a tachykinin receptor antagonist and a serotonin agonist, such as sumatriptan, in combination with xanthan gum, sodium carboxymethylcellulose and microcrystalline cellulose, sucrose, sodium benzoate, flavor, color, and purified water.

U.S. Publication No. 2004/0029927 discloses a syrup formulation that includes sumatriptan and triprolidine hydrochloride in combination with glycerol, liquid sucrose, hydroxyethylcellulose, citric acid, sodium citrate, sodium saccharin, flavor, ethanol, levomenthol, domiphen hydrobromide, color, and water.

Others have reported alternative ways of masking the unpleasant taste of certain pharmaceutically active ingredients. For example, U.S. Pat. No. 6,576,677 describes compositions that include a basic medicament having an unpleasant taste and polyvinylpyrrolidone and/or copolyvidone. The compositions may be in the form of water-soluble liquids, syrups, elixirs, jellies, dry syrups, effervescent preparations, lemonades, aerosols, ophthalmic solutions, nasal drops, suppositories, cataplasmas, liniments, lotions and fine granules. The unpleasant taste of the medicament is reportedly alleviated by adding the polyvinylpyrrolidone and/or copolyvidone in amounts of 5 to 200 parts by weight per 1 part by weight of the medicament.

Sumatriptan succinate is presently commercially available by prescription in injection, nasal spray, and tablet form as a sole active ingredient (Imitrex®, GlaxoSmithKline). Imitrex® is available as tablets for oral administration containing either 35 mg, 70 mg or 140 mg of sumatriptan succinate equivalent to 25, 50, or 100 mg of sumatriptan, respectively, and the following inactive ingredients: croscarmelose sodium, lactose, magnesium stearate, microcrystalline cellulose, and titanium dioxide. Imitrex® injection is a solution of the drug in 0.7% sodium chloride. Imitrex® nasal spray contains the following inactive ingredients: monobasic potassium phosphate, dibasic sodium phosphate, sulfuric acid, sodium hydroxide, and water. The pH of the nasal spray is 5.5.

Because of the ease of preparing solid dosage forms, tablets and capsules are often the preferred dosage form for many drugs including sumatriptan. Indeed, the only oral FDA approved sumatriptan products are in solid form, i.e., as tablets. Liquid dosage forms present more of a challenge because of the unpredictable taste, solubility and stability characteristics of the active compound, as well as the various excipients, in different solvents. In view of the foregoing, it would be desirable to have suitable taste-masked oral liquid sumatriptan compositions with improved taste, dissolution, and stability properties as an additional treatment option.

### SUMMARY OF THE INVENTION

The present invention provides oral liquid compositions that include sumatriptan, or a pharmaceutically acceptable salt or metabolite thereof, and a pharmaceutically acceptable carrier that provides a liquid portion of the composition. The carrier is selected to minimize formation of one or more oxidation impurities, and in a preferred embodiment the composition is substantially free of at least one of the oxidation impurities after storage for at least about one month at 40° C. Preferably, each oxidation impurity is present in an amount of less than about 0.5 percent (w/w) of the sumatriptan, or salt or metabolite thereof, and more preferably all oxidation impurities are present in an amount of less than about 0.5 percent (w/w).

In one embodiment, the carrier includes one or more of a solubilizing agent, thickening agent, sweetening agent, flavoring agent, colorant agent, preservative agent, or antioxidant component. Preferably, the carrier includes glycerin, the sweetening agent is present and includes sorbitol or sucralose, the preservative agent is present and includes a sorbate-containing component, the flavoring agent is present and provides a mint flavor, or the antioxidant component is present and includes a gallate-containing component, or any combination thereof.

In an exemplary embodiment, the sumatriptan, or salt or metabolite thereof, is present in an amount of about 0.5 percent to 5 percent (w/v) of the composition, and the carrier includes glycerin present in an amount of about 5 percent to 60 percent (v/v), sorbitol solution present in an amount up to about 60 percent (w/v), sucralose present in an amount of about 0.1 percent to 1.5 percent (w/v), colorant agent present in an amount of about 0.0001 percent to 0.05 percent (w/v), potassium sorbate present in an amount of about 0.01 percent to 0.5 percent (w/v), peppermint flavor present in an amount of about 0.01 percent to 1 percent (v/v), and propyl gallate present in an amount of about 0.01 to 0.25 percent (w/v).

The present invention also relates to methods of preventing, treating, or managing migraine attacks in a mammal by administering to the mammal a pharmaceutically effective amount of the compositions. The composition is typically administered one to four times a day. The total daily dose of sumatriptan is generally from about 25 mg to 200 mg.

The present invention further encompasses oral liquid compositions that include sumatriptan, or a pharmaceutically acceptable salt or metabolite thereof, and a pharmaceutically acceptable carrier that includes a liquid portion of the composition. The composition can include only a sweetening agent and a flavoring agent, which in a preferred embodiment provides a mint flavor; or the composition can include additional components, preferably without need for a separate taste-masking component. The sweetening agent and flavoring agent are included in amounts sufficient to collectively minimize or mask the bitterness of the sumatriptan.

The mint flavor may include spearmint, peppermint, or a combination thereof. In a preferred embodiment, the sweetening agent includes sucralose. In this embodiment, the sucralose is usually present in an amount of about 0.1 percent to 0.6 percent (w/v) of the composition, and the flavoring agent is generally present in an amount of about 0.01 percent to 1 percent (v/v). The sumatriptan, or salt or metabolite thereof, is typically present in an amount of less than or equal to about 40 mg/mL of sumatriptan base, of the composition.

Preferably, the carrier further includes one or more of a wetting agent, surfactant component, stabilizing agent, solubilizing agent, thickening agent, perfuming agent, colorant agent, preservative agent, or buffering agent. In an exemplary embodiment, the carrier includes glycerin and sorbitol, the sweetening agent is present and includes a sucralose, acesulfame, aspartame or saccharin-containing component, the preservative agent is present and includes a sorbate-containing component, and the buffering agent is present and includes an acetate, phosphate, or citrate-containing buffer.

The present invention further relates to methods of preparing an oral liquid sumatriptan composition. The methods include dissolving an amount of sumatriptan, or a pharmaceutically acceptable salt or metabolite thereof, into at least one liquid adjuvant to form a liquid sumatriptan solution, and combining the liquid sumatriptan solution with a sweetening agent and a flavoring agent that provides a mint flavor to form the oral liquid sumatriptan composition. In one embodiment, the sweetening agent is in liquid form and acts as the liquid adjuvant.

In one embodiment, the methods include adding a bitterness-reducing agent. The at least one liquid adjuvant generally includes water, a buffering agent, or both. Preferably, the oral liquid sumatriptan composition is a clear solution.

The present invention also includes oral liquid compositions that include sumatriptan, or a pharmaceutically acceptable salt or metabolite thereof, and a pharmaceutically acceptable carrier that includes a liquid portion of the composition. The carrier includes a bitterness-reducing agent and a flavoring agent.

In a preferred embodiment, the bitterness-reducing agent is present in an amount of about 0.01 percent to 1 percent (v/v) of the composition, the flavoring agent is present in an amount of about 0.01 percent to 1 percent (v/v) of the composition, or both. The bitterness-reducing agent may be any suitable compound effective in masking the bitter taste of sumatriptan, including monoammonium glycyrrhizinate or menthol, or a combination thereof. Similarly, the flavoring agent may provide any flavor suitable for use in a pharmaceutical composition. Such flavors include chocolate, peppermint, cinnamon, coffee, grape, grapefruit, vanilla, orange, lemon, lime, mango, strawberry, pineapple, cherry, or a combination thereof.

In addition, the present invention is directed to substantially stable oral liquid sumatriptan compositions that include the present compositions, and one or more of the bitterness-reducing agent, the flavoring agent, and a sweetening agent are each present in an amount sufficient to reduce the bitter taste of sumatriptan.

Certain combinations of sweetening and flavoring agents have been discovered to work particularly well. Examples include embodiments where the sweetening agent includes sucralose and the flavoring agent provides a mint flavor, preferably peppermint; the sweetening agent includes acesulfame and the flavoring agent provides an artificial chocolate flavor; or the sweetening agent includes saccharin and the flavoring agent provides a grape flavor.

Preferably, the composition is at least substantially stable and includes sumatriptan succinate. Typically, the degradation of sumatriptan, or the salt or metabolite thereof, over a period of no more than about one month is no more than about 2 percent (w/w) at 50° C.

### BRIEF DESCRIPTION OF THE DRAWINGS

Further features and advantages of the invention can be ascertained from the detailed description that is provided below in connection with the following drawing(s):

FIG. 1 is a graph showing the solubility profile of sumatriptan as a function of pH;

FIG. 2 is a graph showing the degradation of sumatriptan (in percent area by area) in buffers at different pHs and water at different times;

FIG. 3 is a graph showing the percent degradation of sumatriptan in Formulations 1 and 2 at 50° C. in different solvents at different times;

FIG. 4 is a graph showing the percent degradation of sumatriptan in Formulations 3, 3a, 4, and 5 at 50° C. in different solvents at different times;

FIG. 5 is a graph showing the percent degradation of sumatriptan in Formulations 3a, 4, 5, and 6a at 50° C. at different times;

FIG. 6 is a chromatogram showing that the oxidation impurity is not present in solid sumatriptan powder at 4° C.;

FIG. 7 is a chromatogram showing that the oxidation impurity is not present in solid sumatriptan powder at 50° C.;

FIG. 8 is a chromatogram showing formation of the oxidation impurity (with a retention time of 20.871 minutes) upon incubation of sumatriptan succinate in a 25% v/v H₂O₂ solution at room temperature for 2 hours;

FIG. 9 is a chromatogram showing that the oxidation impurity (with a retention time of 21.21 minutes) is present in the Imitrex® injection formulation, and a mass spectrum indicating that the impurity has a molecular weight of 312.1; and

FIG. 10 is a chromatogram showing that the oxidation impurity (with a retention time of 20.90 minutes) is present in the liquid compositions according to the invention, and a mass spectrum indicating that the impurity has a molecular weight of 312.1.

### DETAILED DESCRIPTION OF THE PREFERRED EMBODIMENTS

The present invention provides oral liquid compositions that include the active ingredient sumatriptan, or a pharmaceutically acceptable salt or metabolite thereof, and a pharmaceutically acceptable carrier that includes a liquid portion of the composition. The composition is substantially free of oxidation impurities after storage for about one month, preferably about two months, and more preferably for about three months at 40° C. By “oxidation impurity” is meant any impurity that forms due to oxidation of sumatriptan compositions in liquid form. The oxidation impurity is preferably the oxidation impurity identified in the examples below, ie., the impurity at peak #7. By “substantially free of an oxidation impurity” is meant that less than about 1 weight percent, preferably less than about 0.75 weight percent, and more preferably less than about 0.5 weight percent, based on the weight of sumatriptan, or salt or metabolite thereof, is present. As used herein, “oral liquid composition(s)” include emulsions, solutions, suspensions, concentrates, syrups and elixirs, but do not include solid dosage forms that include minor amounts of liquids therein, such as capsules or tablets. Preferably, “oral liquid composition(s)” mean sumatriptan, or a pharmaceutically acceptable salt or metabolite thereof, in solution.

The present liquid dosage forms provide certain advantages over the solid forms conventionally available. For example, liquid dosage forms are much easier to swallow and typically do not require separate water to administer. Patient compliance is also easier to facilitate with the flavoring and colorant agents that are typically included in liquid dosage forms. This is often an issue with very young patients and the elderly. The present liquid dosage forms further increase patient compliance by providing a dosage form that has reduced bitterness or, preferably, that does not detectably exhibit the bitter taste of sumatriptan, or salts or metabolites thereof, when orally consumed.

Sumatriptan is preferably used in the form of a pharmaceutically acceptable salt or metabolite that retains the biological effectiveness and properties of sumatriptan and is not biologically or otherwise undesirable. As used herein, “sumatriptan” includes the agent itself, as well as its pharmaceutically acceptable salts or an active metabolite unless specifically noted otherwise.

The term “pharmaceutically acceptable salt(s)” or “a pharmaceutically acceptable salt thereof” refers to salt(s) prepared from pharmaceutically acceptable non-toxic acid or bases including inorganic acids and bases and organic acids or bases. The pharmaceutically acceptable salts used in the present invention may be amphoteric, may be present in the form of internal salts, or both.

Sumatriptan may form acid addition salts. Exemplary salts include salts of inorganic or organic acids such as hydrochloride, hydrobromide, sulphate, nitrate, phosphate, formate, mesylate, citrate, benzoate, fumarate, maleate, tartrate, and succinate. A particularly suitable and preferred pharmaceutically acceptable salt of sumatriptan is sumatriptan succinate (1:1) (C₁₄H₂₁N₃O₂S.C₄H₆O₄), which is chemically described as 3-[2-(dimethylamino)ethyl]-N-methyl-indole-5-methanesulfonamide succinate (1:1).

Sumatriptan can be prepared by any suitable method available to one of ordinary skill in the art. For instance, by the methods described in U.S. Pat. Nos. 4,816,470 and 5,037,845, each of which is incorporated by express reference thereto.

An oxidation impurity of sumatriptan, not hereinbefore described or reported in the literature, has now been discovered. The compositions of the present invention advantageously provide liquid formulations that include the oxidation impurity at low levels. Typically, one or more excipients are selected according to the invention to minimize formation of the oxidation impurity in liquid formulations. The oxidation impurity is generally present in an amount of less than about 0.5 percent (w/w), preferably less than about 0.3 percent (w/w), of the sumatriptan, or salt or metabolite thereof. More preferably, the oxidation impurity is present in an amount of less than about 0.2 percent (w/w) of the sumatriptan, or salt or metabolite thereof.

Different agents may be used singly or in combination to form a carrier that provides liquid formulations that are substantially free of the oxidation impurity. In one embodiment, the carrier of the composition includes one or more of a solubilizing agent, thickening agent, sweetening agent, flavoring agent, colorant agent, preservative agent, or antioxidant component. Generally, in this embodiment, the carrier includes glycerin, the sweetening agent includes sorbitol, sucralose, or both, the preservative agent includes a sorbate-containing component, the flavoring agent provides a mint flavor, the antioxidant component includes a gallate-containing component, or any combination thereof. For example, the sumatriptan, or salt or metabolite thereof, is typically present in an amount of about 0.5 percent to 5 percent (w/v) of the composition, and the carrier usually includes glycerin present in an amount of about 5 percent to 60 percent (v/v), sorbitol solution present in an amount of up to about 60 percent (v/v), sucralose present in an amount of about 0.1 percent to 1.5 percent (w/v), colorant agent present in an amount of about 0.0001 percent to 0.05 percent (w/v), potassium sorbate present in an amount of about 0.01 percent to 0.5 percent (w/v), peppermint flavor present in an amount of about 0.01 percent to 1 percent (v/v), and propyl gallate present in an amount of about 0.01 to 0.25 percent (w/v). The sorbitol can be present in an amount of about 15 to 30 percent (v/v) in one preferred embodiment.

While the compositions can generally include any type of carrier, in a preferred embodiment, the compositions are substantially free, more preferably entirely free, of sorbitol, glycerin, polyethylene glycol, or a combination thereof. The compositions may advantageously include propylene glycol, preferably when the compositions have reduced amounts of sorbitol.

The compositions of the present invention also encompass liquid formulations that include a bitterness-reducing agent and flavoring agent. It has now been discovered that including a bitterness-reducing agent (or bitter mask) and flavoring agent in an oral liquid sumatriptan composition provides the surprising and unexpected results of both improved stability and effective taste-masking. In a preferred embodiment, effective taste-masking is achieved without surrounding the sumatriptan with a coating, film, or encapsulant, so that a portion of the drug remains in direct contact with the carrier, and when orally administered, the drug can directly contact the taste buds. In one embodiment, the bitterness-reducing agent is present in an amount of about 0.01 percent to 1 percent (v/v). Preferably, in this embodiment the bitterness-reducing agent is present in an amount of about 0.05 percent to 0.5 percent (v/v), and more preferably is present in an amount of about 0.1 percent to 0.3 percent (v/v). An exemplary amount is about 0.15 percent (v/v). Preferably, the oral liquid compositions of the present invention are achieved while being at least essentially free of a buffering agent, more preferably entirely free of a buffering agent, which is typically required to provide for stable oral liquid pharmaceutical compositions.

The bitterness-reducing agent is selected from any suitable agent that can effectively mask the unpleasant taste of sumatriptan. A preferred bitterness-reducing agent includes monoammonium glycyrrhizinate or menthol, or both. Other suitable bitter masks include Firmenich's N&A Masking Flavor 501483T, Bell Flavors and Fragrances' Bitterness Mask 141.18074, Mafco's Magnasweet™ 110 (2×), and FONA's Sweet-am™ 918.005.

The flavoring agent can enhance patient compliance by making the composition more palatable, particularly in the case of the imbibable compositions of the present invention. The flavoring agent is typically selected in type and amount to decrease or eliminate any bitter taste that would otherwise be detectable by the patient to whom the compositions are administered, particularly when used in conjunction with the bitterness-reducing agent to provide a synergistic reduction in bitterness of the succinate formulations of the invention. The flavoring agent is preferably used in amounts that reduce or minimize any unpleasant aftertaste that may persist after administration.

The flavoring agent is typically present in an amount of about 0.01 percent to 1 percent (v/v), preferably 0.05 percent to 0.75 percent (v/v), and more preferably about 0.1 percent to 0.5 percent (v/v). Examples of a suitable flavoring agent includes one or more of menthol, any mint, anise, chocolate, cinnamon, coffee, vanilla, and any fruit flavor, such as one or more of grape, grapefruit, orange, lemon, lime, mango, strawberry, pineapple, or cherry; or a combination thereof. A flavoring agent including chocolate, peppermint, spearmint, cinnamon, grape, or cherry has been found to work especially well according to the invention. The chocolate flavor provided may be either an artificial dark or milk chocolate flavor, and dark chocolate flavor is also a preferred flavoring agent in one embodiment.

Certain combinations of bitterness-reducing and flavoring agents are preferred for use in the compositions of the present invention. For instance, combining a flavoring agent that provides a cinnamon flavor with a bitterness-reducing agent that includes Mafco's Magnasweet™ 110 (2×) is particularly effective in masking the taste of sumatriptan. The combination of a flavoring agent that provides a cherry flavor with a bitterness-reducing agent that includes FONA's Sweet-am™ 918.005 is also effective.

Furthermore, the present invention relates to oral liquid compositions that include sumatriptan, or a pharmaceutically acceptable salt or metabolite thereof, and a pharmaceutically acceptable carrier that provides a liquid portion of the composition. In preferred embodiments, the carrier as a whole is liquid, e.g., a suspension or solution, preferably a ready-to-use liquid that is substantially free of any precipitates. The carrier in one embodiment includes a sweetening agent and a flavoring agent. It has been surprisingly and unexpectedly discovered that a balance of sweetness and flavor of the carrier for sumatriptan can yield a pleasant-tasting sumatriptan composition. The compositions include a sweetening agent and a flavoring agent each in an amount, or collectively in an amount, suitable to provide a palatable dosage form. Without being bound by theory, it is believed that increasing amounts of flavoring agent and sweetening agent are desired to minimize or mask the bitter taste as the amount of sumatriptan increases. Preferably, this can be achieved in a formulation that is substantially (or preferably entirely) free of any other bitterness-masking agent, although such agents can be included. Advantageously, the sumatriptan is not required to be coated or encapsulated to mask or minimize its bitter taste in the dosage form.

Flavoring agents that provide a mint flavor, which may include spearmint, peppermint, or a combination thereof, have been found to work particularly well with sucralose to provide palatable, stable, oral liquid sumatriptan compositions. In one embodiment, the sucralose is present in an amount of about 0.1 percent to 0.6 percent (w/v) of the composition, and the peppermint flavor is present in an amount of about 0.01 percent to 1 percent (v/v). In an exemplary embodiment, sucralose is present in an amount of about 0.4 percent (w/v) of the composition, and the peppermint flavor is present in an amount of about 0.4 percent (v/v). While other excipients may be included in the carrier, in one embodiment the carrier is substantially free of any other excipients except for sucralose and a flavoring agent.

The compositions of the present invention preferably further include one or more of a wetting agent, surfactant component, stabilizing agent, solubilizing agent, thickening agent, sweetening agent, perfuming agent, colorant agent, preservative agent, buffering agent, or antioxidant component. In the case of each of these pharmaceutically acceptable carriers, any suitable type or amount of carrier available to those of ordinary skill in the art may be included according to the invention, so long as it does not adversely effect the taste-masking or stability characteristics of the compositions. Although a composition including a buffering agent or salt may be suitable, preferably, the composition is essentially free of, more preferably entirely free of, a buffering agent. In an embodiment where the composition is essentially free or entirely free of a buffering agent, the possible range of buffer is replaced by the same range of water or other suitable liquid adjuvant. Sumatriptan may be dissolved or suspended in a solvent such as water, vegetable oil, alcohol, polyethylene glycol, propylene glycol or glycerin. Preferably, the solvent includes water and/or glycerin, and is substantially free of vegetable oil. By “substantially free” or “essentially free” it is meant that less than about 2 weight percent, preferably less than about 1 weight percent, and more preferably less than about 0.5 weight percent of the component, by weight of the composition, is present in the composition. In a preferred embodiment, substantially or essentially free refers to less than about 0.01 weight percent, preferably less than about 0.001 weight percent.

A wetting agent or surfactant component can be included in the liquid compositions of the present invention that, when used, includes one or more quaternary ammonium compounds, such as benzalkonium chloride, benzethonium chloride and cetylpyridinium chloride; TPGS, dioctyl sodium sulfosuccinate; polyoxyethylene alkylphenyl ethers, such as nonoxynol 9, nonoxynol 10, and octoxynol 9; poloxamers (polyoxyethylene and polyoxypropylene block copolymers); polyoxyethylene fatty acid glycerides and oils, such as polyoxyethylene (8) caprylic/capric mono- and diglycerides (e.g., Labrasol™, Gattefosse), polyoxyethylene (35) castor oil and polyoxyethylene (40) hydrogenated castor oil; polyoxyethylene alkyl ethers, such as polyoxyethylene (20) cetostearyl ether; polyoxyethylene fatty acid esters, such as polyoxyethylene (40) stearate; polyoxyethylene sorbitan esters, such as polysorbate 20 and polysorbate 80 (e.g., Tween™ 80, ICI); propylene glycol fatty acid esters, such as propylene glycol laureate (e.g., Lauroglycol™, Gattefosse); sodium lauryl sulfate; fatty acids and salts thereof, such as oleic acid, sodium oleate and triethanolamine oleate; glyceryl fatty acid esters, for example glyceryl monostearate; sorbitan esters, such as sorbitan monolaurate, sorbitan monooleate, sorbitan monopalmitate and sorbitan monostearate; tyloxapol; lecithin; stearyl triethanolamine; laurylaminopropionic acid; and mixtures thereof. Such surfactant component or wetting agent, if present, will typically together form about 0.25 weight percent to about 15 weight percent, preferably about 0.4 weight percent to about 10 weight percent, and more preferably about 0.5 weight percent to about 5 weight percent, of the total weight of the composition.

A thickening agent or viscosity-enhancing agent can be included to generally thicken the liquid composition, which typically improves the mouth-feel of the composition, and/or to help coat the lining of the gastrointestinal tract. While any suitable thickening agent can be included in the compositions of the present invention, a preferred thickening agent, when used, includes one or more of acacia, alginic acid bentonite, carbomer, carboxymethylcellulose calcium or sodium, cetostearyl alcohol, methyl cellulose, ethylcellulose, glycerin, gelatin guar gum, hydroxyethylcellulose, hydroxypropylcellulose, hydroxypropylmethylcellulose, maltodextrin, polyvinyl alcohol, povidone, propylene carbonate, propylene glycol alginate, sodium alginate, sodium starch glycolate, starch tragacanth, and xanthan gum, and any combination thereof. More preferred thickening agents are glycerin, hydroxypropylmethylcellulose, and xanthan gum, and any combination thereof. Such a thickening agent, if present, will typically form about 0.1 weight percent to 20 weight percent, preferably about 0.3 weight percent to about 15 weight percent, and more preferably about 0.5 weight percent to 4 weight percent, of the total weight of the composition.

A sweetening agent, such as one or more of sorbitol, saccharin, sucralose, sodium saccharin, xylitol, glycerin, sucrose, aspartame, fructose, neotame, sodium saccharate, glycyrrhizin dipotassium, acesulfame potassium, mannitol, propylene glycol, invert sugar, and mixtures thereof, whether in solid form or liquid form (e.g., liquid sugar or sucrose) can be added to modify the taste of the composition by reducing the bitterness thereof and to increase the stability of the sumatriptan oral liquid composition. Preferably, the sweetening agent includes a sweetener that provides a high level of sweetness at a low concentration, such as sucralose, acesulfame, or a saccharin-containing component, or a combination thereof, more preferably sucralose or acesulfame, or a combination thereof. Bulk sweeteners that need higher concentrations, but provide a lower level of sweetness, such as sorbitol, mannitol, sucrose, and glucose, are not generally as suitable. The acesulfame-containing component is preferably acesulfame potassium, while the saccharin-containing component is preferably sodium saccharin. Alternatively or in addition, a viscous sweetener, such as one or more of a sorbitol solution, syrup (sucrose solution), glycerin, or high-fructose corn syrup can be used and, in addition to sweetening effects, can also be useful to increase viscosity and to retard sedimentation. Such a sweetening agent, if present, will typically be present in an amount sufficient to reduce the bitterness of the sumatriptan, and preferably to also mask any other off-flavor components included in the formulation. By “reduce the bitterness” is typically meant that the bitterness is minimized, generally in a manner detectable by a consumer. Preferably, the bitterness of sumatriptan is reduced sufficiently to mask the bitter flavor from consumer detection. Typical amounts of sweetening agent include about 0.05 weight percent to about 10 weight percent, preferably about 0.1 weight percent to about 5 weight percent, more preferably about 0.5 weight percent to 2 weight percent, of the total weight of the composition.

A colorant agent, when included, can provide the compositions with a more aesthetic and/or distinctive appearance. Colorant agents preferable for inclusion in the present invention include one or more water-soluble synthetic organic food additives (e.g., food dyes such as food red dye Nos. 2 and 3, food yellow dye Nos. 4 and 5 and food blue dye Nos. 1 and 2), water-insoluble lake dyes (e.g., aluminum salts of the above water-soluble synthetic organic food additives, etc.), and natural pigments (e.g., beta-carotene, chlorophyll, iron oxide red, etc.). Suitable colorants include D&C Red No. 33, FD&C Red No. 3, FD&C Red No. 40, D&C Yellow No. 10, and C Yellow No. 6. Such a colorant agent, if present, will typically form about 0.001 weight percent to about 1 weight percent, preferably about 0.001 weight percent to about 0.5 weight percent, and more preferably about 0.0075 weight percent to about 0.25 weight percent, of the total weight of the composition.

Examples of an optional, but preferred, preservative component includes sodium benzoate, butylated hydroxy toluene, butylated hydroxyanisole, ethylenediamine tetraacetic acid, paraoxybenzoic acid esters (e.g., methyl paraben), chlorobutanol, benzyl alcohol, phenylethylalcohol, dehydroacetic acid, sorbic acid, potassium sorbate, benzalkonium chloride, benzethonium chloride, phenol, phenylmercuric nitrate, thimerosal, and mixtures thereof. A preferred preservative component for use in the present invention includes potassium sorbate or methyl paraben, or both. More preferably potassium sorbate is included in the preservative component. A preservative can be added to the compositions at levels safe for ingestion to improve storage stability. A preservative component, if present, will typically form about 0.01 weight percent to about 5 weight percent, preferably about 0.05 weight percent to about 3 weight percent, and more preferably about 0.1 weight percent to about 2 weight percent, of the total weight of the composition.

In one embodiment, a buffering agent is preferably included in the liquid compositions and can include a buffer solution of one or more gluconate, lactate, citrate, acetate, phosphate, and/or carbonate salts. The buffering agent typically provides a pH of about 5 or higher and can also modulate drug solubility. Preferably, the buffering agent includes a citrate or an acetate, such as sodium salt. Such a buffering agent, if present, will typically form about 10 weight percent to about 80 weight percent, preferably about 30 weight percent to about 70 weight percent, and more preferably about 40 weight percent to about 60 weight percent, of the total weight of the composition. In another embodiment, however, which is just as (if not more) preferred, the composition is essentially free of, and more preferably entirely free of, a buffering agent.

Examples of a suitable antioxidant component, if used, include one or more of the following: sulfites; ascorbic acid; ascorbates, such as sodium ascorbate, calcium ascorbate, or potassium ascorbate; ascorbyl palmitate; fumaric acid; ethylene diamine tetraacetic acid (EDTA); tocopherol; gallates, such as propyl gallate, octyl gallate, or dodecyl gallate; vitamin E; and mixtures thereof. The antioxidant component provides long term stability to the liquid compositions. Addition of the antioxidant component can help enhance and ensure the stability of the compositions and renders the compositions stable even after three months at 40° C. A suitable amount of the antioxidant component, if present, is about 0.01 weight percent to about 3 weight percent, preferably about 0.05 weight percent to about 2 weight percent, of the total weight of the composition. A more preferred amount of antioxidant, if present, may be about 0.25 weight percent to about 1 weight percent of the total weight of the composition.

Solubilizing and emulsifying agents can be included to facilitate more uniform dispersion of the active ingredient or other excipient that is not generally soluble in the liquid carrier. Examples of a suitable emulsifying agent, if used, includes, for example, gelatin, egg yolk, casein, cholesterol, acacia, tragacanth, chondrus, pectin, methyl cellulose, carbomer, cetostearyl alcohol, cetyl alcohol, and mixtures thereof. Examples of a suitable solubilizing agent include glycol, glycerin, D-mannitol, trehalose, benzyl benzoate, ethanol, trisaminomethane, cholesterol, triethanolamine, sodium carbonate, sodium citrate, sodium salicylate, sodium acetate, and mixtures thereof. Preferably, the solubilizing agent includes glycerin. The solubilizing or emulsifying agent is/are generally present in an amount sufficient to dissolve or disperse the sumatriptan in the carrier. Typical amounts when a solubilizing or an emulsifier are included are from about 1 weight percent to about 80 weight percent, preferably about 20 weight percent to about 65 weight percent, and more preferably about 25 weight percent to about 55 weight percent, of the total weight of the composition.

A suitable isotonizing agent, if used, includes sodium chloride, glycerin, D-mannitol, D-sorbitol, glucose, and mixtures thereof. A suitable amount of the isotonizing agent, when included, is typically about 0.01 weight percent to about 15 weight percent, more preferably about 0.3 weight percent to about 4 weight percent, and more preferably about 0.5 weight percent to about 3 weight percent, of the total weight of the composition.

Through selection and combination of excipients according to the invention, liquid sumatriptan compositions can be provided that exhibit sufficiently reduced bitterness coupled with sufficient stability, preferably without need for a buffering agent, all while retaining the expected and desired performance with respect to drug concentration, dissolution, dispersion, stability, safety, emulsification, efficacy, flavor, patient compliance, and/or other pharmacokinetic, chemical and/or physical properties that exist in solid or other non-oral liquid dosage forms.

It has now been further discovered that a carrier that includes one or more of a solubilizing agent, sweetening agent, or preservative agent, in combination with the bitterness-reducing agent and flavoring agent, is particularly advantageous in an oral liquid sumatriptan composition. Preferably, two or more of the solubilizing agent, sweetening agent, or preservative agent, will be included. In one more preferred embodiment, the solubilizing agent, sweetening agent, and preservative agent are all included along with the sumatriptan.

To provide a therapeutically or prophylactically effective amount of the active ingredient, sumatriptan, expressed as its base, is generally present in an amount of about 1 mg/mL to 50 mg/mL of the composition, preferably about 5 mg/mL to 35 mg/mL, and more preferably about 10 mg/mL to 30 mg/mL. The concentrations expressed herein are based on equivalents of sumatriptan base. Typically, sumatriptan is present in an amount of less than or equal to about 40 mg/mL of sumatriptan base. The phrase “therapeutically effective amount” means that amount of sumatriptan that provides a therapeutic benefit in the treatment or management of migraine and general malaise associated therewith. The term “prophylactically effective amount” means that amount of sumatriptan that, alone or with another active ingredient, inhibits or prevents migraine and general malaise associated therewith.

The amounts of the other components in the composition may vary. Generally, the glycerin can be present in an amount of about 5 percent to 60 percent (v/v) and the sorbitol is optionally present in an amount of about up to about 60 percent (v/v). Typically, the sweetening agent is present in an amount of about 0.05 percent to about 2 percent (w/v), while the preservative agent is present in an amount of about 0.01 percent to 1 percent (w/v). In one preferred embodiment, all four of these components are included in the liquid sumatriptan compositions. The buffering agent, if present as in one embodiment, is generally present in an amount of about 50 percent to 95 percent (v/v). The buffering agent preferably maintains and provides a pH of about 3 to 7 when included. As used herein, the terms (v/v) and (w/v) refer to percentages based on volume and percentages based on weight per volume, respectively.

In one embodiment, the carrier is substantially water soluble, and preferably entirely water soluble. By “substantially water soluble” is meant that at least about 85 percent by weight or volume of the carrier dissolves in water, preferably at least about 90 percent, and more preferably at least about 95 percent. In another embodiment, the sumatriptan compositions of the present invention are substantially cellulose-free, i.e., substantially free of any cellulose-based pharmaceutically acceptable excipients. Such excipients include methylcellulose, ethylcellulose, carboxymethylcellulose, hydroxyethylcellulose, hydroxypropylcellulose, hydroxypropylmethylcellulose, and microcrystalline cellulose.

It has further been discovered that certain combinations of sweetening and flavoring agents supply exemplary taste-masking characteristics to the compositions of the present invention. The following combinations have been found to work especially well: (1) the sweetening agent includes a sucralose-containing agent and the flavoring agent provides a mint flavor, preferably a peppermint flavor; (2) the sweetening agent includes an acesulfame-containing component and the flavoring agent provides an artificial chocolate flavor; (3) the sweetening agent includes a saccharin-containing component and the flavoring agent provides a grape flavor. In one preferred embodiment, the sweetening agent includes sucralose in an amount of about 0.4 percent (w/v), the flavoring agent provides a peppermint flavor, and the bitterness-reducing agent includes Firmenich's N&A Masking Flavor 501483T. In another preferred embodiment, the sweetening agent includes acesulfame potassium in an amount of about 0.5 percent to 1 percent (w/v), the flavoring agent provides an artificial chocolate flavor, and the bitterness-reducing agent includes Bell Flavors and Fragrances' Bitterness Mask 141.18074. In yet another preferred embodiment, the sweetening agent includes saccharin sodium present in an amount of about 1 percent (w/v), the flavoring agent provides a grape flavor, and the bitterness-reducing agent includes monoammonium glycyrrhinate.

In another embodiment where a buffer is included, the sumatriptan is present in an amount of about 2.5 mg/mL to 30 mg/mL of sumatriptan base, of the composition, and the carrier includes glycerin present in an amount of about 5 percent to 60 percent (v/v), sorbitol optionally present in an amount of up to about 60 percent (v/v), the sweetening agent (i.e., not sorbitol when it is present) present in an amount of about 0.1 percent to 1.5 percent (w/v), the preservative agent present in an amount of about 0.01 percent to 0.5 percent (w/v), and the buffering agent present in an amount of about 50 percent to 80 percent (v/v). In an exemplary embodiment, the sumatriptan is present in an amount of about 25 mg/mL of sumatriptan base, the glycerin is present in an amount of about 20 percent (v/v), the sorbitol is present in an amount of about 20 percent (v/v), the sweetening agent is present in an amount of about 0.2 percent to 1.5 percent (w/v), and the preservative agent is present in an amount of about 0.1 percent (w/v).

The compositions of the present invention are preferably at least substantially stable, more preferably stable. Preferably, sumatriptan is present as the succinate salt. By “substantially stable” is meant a percent degradation of sumatriptan in the composition over a period of about one month that is no more than about 3 percent, preferably no more than about 2 percent, and more preferably no more than about 1.5 percent, at 50° C. In an exemplary embodiment, the percent degradation of sumatriptan over a period of about one month is no more than about 1 percent to 2 percent at 50° C. In another embodiment, the degradation of sumatriptan over a period of about two months is no more than about 1 percent at 40° C. As used herein, “percent degradation” is meant the value calculated by dividing the sum of degradation peak areas by the sumatriptan peak area and multiplying by 100.

The compositions of the present invention may be packaged in any suitable container, such as HDPE bottle packs, PET, or amber glass bottle packs. Preferably, HDPE bottle packs to facilitate stability of the compositions therein.

The present invention also relates to methods of preparing oral liquid sumatriptan compositions. Typically, the method can include dissolving an amount of sumatriptan, preferably in a therapeutically or prophylactically effective amount, into at least one liquid adjuvant to form a liquid sumatriptan solution and combining the liquid sumatriptan solution with a sweetening agent and flavoring agent that preferably provides a mint flavor to form the oral liquid sumatriptan composition. In one embodiment, the method further includes adding a bitterness-reducing agent, which can substitute for the flavoring agent although is preferably included in combination with a flavoring agent. While a buffering agent can be included in one embodiment, in another the composition is essentially free or entirely free of a buffering agent.

The at least one liquid adjuvant may be selected from any of the agents already described above. In one embodiment, the at least one liquid adjuvant includes water or is water. In another embodiment, the at least one liquid adjuvant includes a buffering agent, or both a buffering agent and water. In a preferred embodiment, the oral liquid sumatriptan composition is a clear solution. By “clear” is meant that the light transmission through the composition is typically at least about 70 percent, preferably at least about 90 percent, and more preferably at least about 95 percent. In an exemplary embodiment, the composition is substantially transparent to the naked eye.

The present invention also provides methods of preventing, treating, or managing migraine attacks in a mammal by orally administering the sumatriptan in liquid form. As used herein, the terms “preventing, treating, or managing” cover preventing, treating, or managing the specified disease in a mammal, particularly a human, and includes: (i) preventing the disease from occurring in a subject that may be predisposed to the disease but has not yet been diagnosed as having it; (ii) inhibiting the disease, i.e., arresting its development before or after it afflicts a patient; or (iii) relieving the disease, i.e., causing regression of the disease. As used herein, “mammal” is meant the class of warm-blooded vertebrate animals that have, in the female, milk-secreting organs for feeding the young. Mammals include humans, apes, many four-legged animals, whales, dolphins, and bats. It should also be understood that symptoms of any disease are also encompassed within the term “managed,” such that managing hypertension may address some or all of the symptoms thereof with or without actually affecting the underlying disease itself.

Sumatriptan has therapeutic applicability for use in the treatment of migraine and associated conditions, for example cluster headache, chronic paroxysmal hemicrania, headache associated with vascular disorders, tension headache and paediatric migraine. As used herein, “migraine” is meant to include migraine without aura, migraine with aura, migraine with typical aura, migraine with prolonged aura, familial hemiplegic migraine, basilar migraine, migraine aura without headache, migraine with acute onset aura, ophthalmoplegic migraine, retinal migraine, childhood periodic syndromes that may be precursors to or associated with migraine, benign paroxysmal vertigo of childhood, alternating hemiplegia of childhood, status migrainosus, and migrainous infarction.

The methods of the invention include administering to a mammal a therapeutically or prophylactically effective amount of an oral liquid sumatriptan composition that includes sumatriptan, or a pharmaceutically acceptable salt or metabolite thereof as that term is defined herein, and at least one pharmaceutically acceptable carrier that includes a bitterness-reducing agent and flavoring agent. The prophylactically or therapeutically effective amount of sumatriptan will vary depending on the subject being treated, the severity of the disease state and the manner of administration, and may be determined routinely by one of ordinary skill in the art. The dose, and perhaps dose frequency, will also vary according to the age, body weight, and response of the individual agent. In general, the total daily dose range is from about 25 mg to 200 mg administered in single or divided doses orally. The composition may typically be administered one to four times a day. It may be necessary to use dosages outside the above ranges in some cases, as will be apparent to those of ordinary skill in the art. Further, it is noted that the clinician or treating physician will know how and when to interrupt, adjust, or terminate therapy in conjunction with individual patient response.

The methods of the invention also contemplate the addition of one or more therapeutic agents with the sumatriptan to provide an additive, more complete, or synergistic effect in preventing, treating, or managing a disease as noted herein. The additional “therapeutic agents,” which can be prophylactic or therapeutic, may be administered in any dosage form(s) suitable for the formulation as are well known in the art. Such dosage forms include solid dosage forms, such as tablets, capsules, powders, and cachets, or liquid dosage forms, such as suspensions, syrups, solutions, and elixirs. The agent may be incorporated in the sumatriptan liquid composition or may be administered in a separate dosage form. The dosage form containing the additional agent to be administered will, in any event, contain a quantity of the additional therapeutic agent (s) in an amount effective to prevent, treat, or alleviate the condition(s) or symptom(s) of the subject being treated. The selection of any such additional therapeutic agents will depend upon the specific disease state being treated, and are described in detail below. Preferably, all active ingredients will be in an oral liquid form, more preferably in a combined form to facilitate patient compliance.

Typical drugs for use in combination with sumatriptan include one or more tachykinin receptor antagonists; antihistaminic and anti-allergenic agents, e.g., chlorpheniramine maleate, diphenhydramine, terphenidine, flunarizine, and cetirizine; sympathomimetics, e.g., phenylpropanolamine pseudoephedrine; anti-emetic and/or gastroprokinetic agents; analgesic and anti-inflammatory agents, e.g., naproxen sodium and paracetamol; and beta-adrenergic blocking agents.

Suitable tachykinin receptor antagonists include (R)-3-(1H-indol-3-yl)-1-[N-(2-methoxybenzyl)acetylano]-2-[N-(2-(4-(piperid in-1-yl)piperidin-1 yl)acetyl)amino]propane). It has been reported that tachykinin receptor antagonists in combination with a serotonin agonist, such as sumatriptan, or a selective serotonin reuptake inhibitor, may be used to treat or prevent psychiatric disorders, pain or nociception, and symptoms of the common cold or allergic rhinitis.

Suitable anti-emetic and/or gastroprokinetic agents include alizapride, alosetron, azasetron, batanopride, bemesetron, benzquinamide, bietanautine, bromopride, buclizine, chlorpromazine, cinitapride, cisapride, clebopride, cyclizine, dimenhydrinate, diphenidol, dolasetron, domperidone, dronabinol, fedotozine, fludorex, flumeridone, galdansetron, granisetron, itasetron, loxiglumide, lurosetron, meclizine, methallatal, metoclopramide, metopimazine, nabilone, naboctate, ondansetron, oxypendyl, palonsetron, pancopride, pipamazine, prochlorperazine, promethazine, scopolamine, sulpiride, thiethylperazine, thioproperazine, trimethobenzamide, tropisetron, zacoprid, and their pharmaceutical acceptable salts or solvates, and any combination thereof.

Analgesics may be employed to alleviate pain in a patient. Thus, the combinations may be used as a preemptive analgesic to treat acute pain such as musculoskeletal pain, post operative pain and surgical pain, chronic pain such as chronic inflammatory pain (e.g., rheumatoid arthritis (RA) and osteoarthritis (OA), neuropathic pain (e.g., post herpetic neuralgia (PHN), trigeminal neuralgia, neuropathies associated with diabetes and sympathetically maintained pain) and pain associated with cancer and fibromyalgia. The combinations may also be used in the treatment or prevention of pain associated with Functional Bowel Disorders (e.g., Irritable Bowel Syndrome), non-cardiac chest pain, and non ulcer dyspepsia.

Suitable analgesic agents include one or more adenosine A1 receptor agonists, opioids, para-aminophenol derivatives and non-steroidal anti-inflammatory drugs (NSAIDs). A1 receptor agonists have been described in the art and include compounds described in the following published applications W0 99/24449, W0 99/24450, W0 99/24451, W0 97/43300, W0 98/16539, W0 98/04126, W0 98/01459, EP 0322242, GB 2226027, EP 222330, W0 98/08855, W0 94/0707 and W0 99/67262. Opioids include alfentanil, buprenorphine, codeine, dextropropoxyphene, diamorphine, dihydrocodeine, fentanyl, methadone, morphine, oxycodone, levorphanol, pentazocine, pethidine, nefopam, flupirtin, meptazinol and tramadol. Para-aminophenol derivatives include paracetamol (also known as acetaminophen), propacetamol, phenacetin, and acetanilide. NSAIDs include naproxen, ibuprofen, flurbiprofen, ketoprofen, dexketoprofen, fenoprofen, fenbufen, tolfenamic acid, mefenamic acid, tiaprofenic acid, indomethacin, oxaprozin, diclofenac, aceclofenac, sulindac, ketorolac, nabumetone, phenylbutazone, azapropazone, diflunisal, piroxicam, tenoxicam, salicylates such as aspirin, and COX-2 inhibitors such as celecoxib (Celebrex™), rofecoxib (Vioxx™), valdecoxib (Bextra™), parecoxib, deracoxib, lumiracoxib (Prexige™, Novartis), BMS347070 (Bristol Myers Squibb), tiracoxib, ABT963 (Abbott), CS502 (Sankyo), GW406381 (GlaxoSmithKline), JTE-522 (Japan Tobacco), etoricoxib (MK663) (Arcoxia™), nimesulide, flosulide, COX189, etodolac, meloxicam, DFP, NS398, L-745337, 2-(4-ethoxy-phenyl)-(4-methanesulfonyl-phenyl)-pyrazolo[1,5-b]pyridazine, 8-acetyl-3-(4-fluorophenyl)-2-(4-methanesulfonylphenyl)-imidazo[1,2-a]pyridine, 4-[2-(3-fluorophenyl)-6-trifluoromethyl-pyrazolo[1,5-a]pyridin-3-yl]-benzenesulfonamide, N-isobutyl-4-[4-(methylsulfonyl)phenyl]-6-(trifluoromethyl)pyrimidin-2-amine, 4-(4-cyclohexyl-2-methyloxazol-5-yl)-2-fluorobenzenesulfonamide, 2-(3,5-difluorophenyl)-3-(4-(methylsulfonyl)phenyl)-2-cyclopenten-1-one, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)-phenyl]-3(2H)-pyridazinone, 2-[(2,4-dichloro-6-methylphenyl)amino]-5-ethyl-benzeneacetic acid, (3Z)-3-[(4-chlorophenyl)[4-(methylsulfonyl)phenyl]methylene]dihydro-2(3H)-furanone, and (S)-6,8-dichloro-2-(trifluoromethyl)-2H-1-benzopyran-3-carboxylic acid.

Suitable beta adrenergic blocking agents are known in the art, including acebutolol, atenolol, betaxolol, bioprolol, carteolol, labetalol, metoprolol, nadolol, penbutolol, pindolol, propanolol, and timolol, and any combination thereof, and are employed in migraine preventive therapy.

The term “about,” as used herein, should generally be understood to refer to both numbers in a range of numerals. Moreover, all numerical ranges herein should be understood to include each whole integer within the range.

Each of the patent applications, patents, publications, and other published documents mentioned or referred to in the Detailed Description is incorporated herein in its entirety by express reference thereto, to the same extent as if each individual patent application, patent, publication, and other published document was specifically and individually indicated to be incorporated by reference.

### EXAMPLES

The invention is further defined by reference to the following examples, describing in detail the methods used to prepare the compositions of the present invention. It will be apparent to those of ordinary skill in the art that many modifications, both to materials and methods, may be practiced without departing from the purpose and interest of the invention. These examples are for illustrative purposes only, and are not to be construed as limiting the appended claims.

### Example 1

### Solubility Studies for Sumatriptan Succinate According to the Invention

The apparent solubility of sumatriptan succinate at room temperature was evaluated for buffers at pHs of 2, 5, 7, and 9. By apparent solubility is meant the solubility of the sumatriptan determined visually. The materials used were sumatriptan succinate and various reagents for preparation of the buffers. Hydrochloric acid was used for preparation of the pH 2 buffer, acetate for preparation of the pH 5 buffer, phosphate for preparation of the pH 7 buffer, and carbonate for the pH 9 buffer. The concentration of the buffers was 0.1 M.

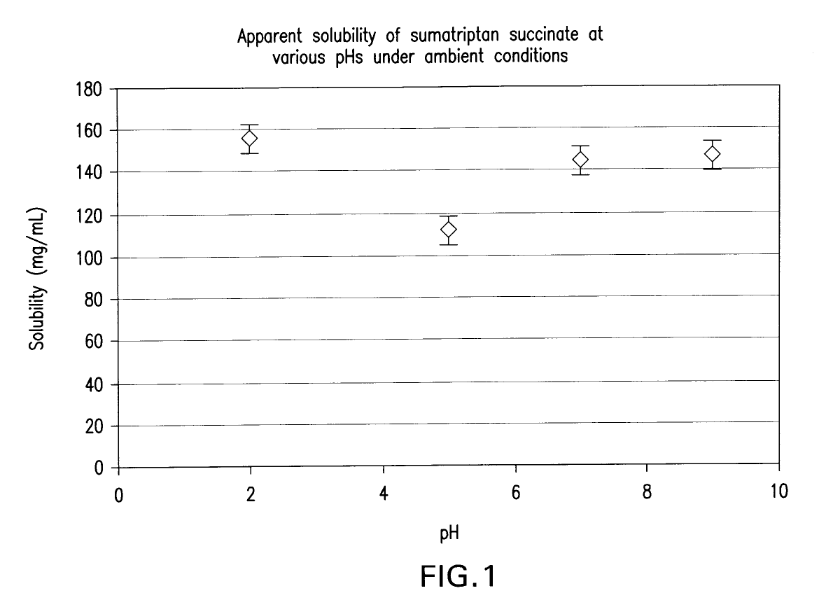

A 1 mL aliquot of each buffer was placed in a glass scintillation vial. To this volume was added a small amount of sumatriptan. Each amount of sumatriptan was weighed on a balance. Weights typically ranged from between 5 and 35 mg. After each new addition of sumatriptan, the vial was shaken and visually inspected to determine if all of the sumatriptan was in solution. When small particles of undissolved sumatriptan appeared, no further amount of sumatriptan was added. These amounts of sumatriptan added before the final addition was added together and were listed as the lower limit of each pH. The upper limit of solubility was taken to be the sum total of sumatriptan added to the buffer when undissolved sumatriptan appeared. The results are provided in Table 1 below. FIG. 1 graphically shows the apparent solubility of sumatriptan as a function of pH at room temperature. The diamond symbol in FIG. 1 represents the average value between the lower and upper limit.

TABLE 1

| pH of Buffer | Lower Limit (mg/mL) | Upper Limit (mg/mL) |
|---|---|---|
| 2 | 149.2 | 162.1 |
| 5 | 109.9 | 119.85 |
| 7 | 138.5 | 150.8 |
| 9 | 138.5 | 140.5 |

As can be seen from the results of Table 1, the apparent solubility of sumatriptan succinate is high at all the tested pH levels. Indeed, the results show that sumatriptan succinate has an apparent solubility of about 100 mg/mL to 115 mg/mL at pHs of 2-9, which indicates that an oral solution of sumatriptan at an amount of 25 mg/mL is attainable.

### Example 2

### Stability Studies for Sumatriptan Succinate According to the Invention

### Example 2.1

### Stability of Sumatriptan Succinate

The physical and chemical stability of sumatriptan succinate was evaluated for one month in water and in the buffers of pH 2, 5, 7, and 9 described previously, at 50° C. and ambient temperature.

To prepare each sample, 20 mg of sumatriptan succinate was weighed out and dissolved in 20 mL of the appropriate buffer or water. After mixing thoroughly, a 1 mL aliquot was removed for high performance liquid chromatography (HPLC) analysis. The remaining volume was then split into a glass tube to be kept at room temperature, and another glass tube to be kept at 50° C. Samples were pulled periodically from the 50° C. tubes for one month and analyzed by HPLC. The results of these studies for each tested pH level at 50° C. are presented below in Tables 2-6.

TABLE 2
(50° C.)

| | Sumatriptan Amount at | Degradation (Percent |
|---|---|---|
| Time (Hours) | pH of 2 (mg/mL) | Area by Area) |
| 0 | 1.02, 0.973 | 0, 0 |
| | Ave. = 0.9965 | Ave. = 0 |
| | Std. Dev. = 0.033 | Std. Dev. = 0 |
| 96 | 1.036, 1.009 | 0.38, 0.43 |
| | Ave. = 1.0225 | Ave. = 0.4 |
| | Std. Dev. = 0.019 | Std. Dev. = 0.025 |
| 264 | 1.044, 1.008 | 0.17, 0.18 |
| | Ave. = 1.026 | Ave. = 0.2 |
| | Std. Dev. = 0.025 | Std. Dev. = 0.005 |
| 432 | 1.028, 0.991 | 0.3, 0.3 |
| | Ave. = 1.0095 | Ave. = 0.3 |
| | Std. Dev. = 0.026 | Std. Dev. = 0 |
| 768 | 0.99, 0.953 | 1.81, 1.89 |
| | Ave. = 0.9715 | Ave. = 1.9 |
| | Std. Dev. = 0.026 | Std. Dev. = 0.04 |

TABLE 3
(50° C.)

| | Sumatriptan Amount at | Degradation (Percent |
|---|---|---|
| Time (Hours) | pH of 5 (mg/mL) | Area by Area) |
| 0 | 1.041, 1.025 | 0, 0.01 |
| | Ave. = 1.033 | Ave. = 0.005 |
| | Std. Dev. = 0.011 | Std. Dev. = 0.005 |
| 96 | 1.097, 1.025 | 0.01, 0.01 |
| | Ave. = 1.033 | Ave. = 0.01 |
| | Std. Dev. = 0.029 | Std. Dev. = 0 |
| 264 | 1.111, 1.061 | 0.03, 0.04 |
| | Ave. = 1.086 | Ave. = 0.035 |
| | Std. Dev. = 0.035 | Std. Dev. = 0.005 |
| 432 | 1.055, 0.973 | 0.4, 0.6 |
| | Ave. = 1.014 | Ave. = 0.5 |
| | Std. Dev. = 0.058 | Std. Dev. = 0.1 |
| 768 | 0.999, 0.928 | 2.35, 2.4 |
| | Ave. = 0.9635 | Ave. = 2.4 |
| | Std. Dev. = 0.05 | Std. Dev. = 0.1 |

TABLE 4
(50° C.)

| | Sumatriptan Amount at | Degradation (Percent |
|---|---|---|
| Time (Hours) | pH of 7 (mg/mL) | Area by Area) |
| 0 | 0.958, 1.001 | 0, 0.01 |
| | Ave. = 0.9795 | Ave. = 0.005 |
| | Std. Dev. = 0.03 | Std. Dev. = 0.005 |
| 96 | 1.014, 1.036 | 0.17, 0.28 |
| | Ave. = 1.025 | Ave. = 0.2 |
| | Std. Dev. = 0.016 | Std. Dev. = 0.055 |
| 264 | 1.005, 1.025 | 1.13, 0.87 |
| | Ave. = 1.015 | Ave. = 1.0 |
| | Std. Dev. = 0.014 | Std. Dev. = 0.13 |
| 432 | 0.952, 0.979 | 1.4, 1.5 |
| | Ave. = 0.9655 | Ave. = 1.45 |
| | Std. Dev. = 0.019 | Std. Dev. = 0.05 |
| 768 | 0.918, 0.942 | 3.57, 3.63 |
| | Ave. = 0.93 | Ave. = 3.6 |
| | Std. Dev. = 0.017 | Std. Dev. = 0.03 |

TABLE 5
(50° C.)

| | Sumatriptan Amount at | Degradation (Percent |
|---|---|---|
| Time (Hours) | pH of 9 (mg/mL) | Area by Area) |
| 0 | 1.013, 1.026 | 0.03, 0 |
| | Ave. = 1.0195 | Ave. = 0.02 |
| | Std. Dev. = 0.009 | Std. Dev. = 0.015 |
| 96 | 1.039, 1.039 | 0.06, 0.06 |
| | Ave. = 1.039 | Ave. = 0.06 |
| | Std. Dev. = 0 | Std. Dev. = 0 |
| 264 | 1.045, 1.048 | 0.11, 0.11 |
| | Ave. = 1.0465 | Ave. = 0.1 |
| | Std. Dev. = 0.002 | Std. Dev. = 0 |
| 432 | 1.005, 1.011 | 1.6, 3.2 |
| | Ave. = 1.008 | Ave. = 2.4 |
| | Std. Dev. = 0.004 | Std. Dev. = 0.8 |
| 768 | 0.0965, 0.982 | 6.16, 5.24 |
| | Ave. = 0.9735 | Ave. = 5.7 |
| | Std. Dev. = 0.012 | Std. Dev. = 0.46 |

TABLE 6
(50° C.)

| | Sumatriptan Amount in | Degradation (Percent |
|---|---|---|
| Time (Hours) | water (mg/mL) | Area by Area) |
| 0 | 1.006, 0.875 | 0, 0 |
| | Ave. = 0.9405 | Ave. = 0 |
| | Std. Dev. = 0.093 | Std. Dev. = 0 |
| 96 | 1.002, 1.082 | 0.02, 0.01 |
| | Ave. = 1.052 | Ave. = 0.02 |
| | Std. Dev. = 0.042 | Std. Dev. = 0.005 |
| 264 | 1.023, 1.103 | 0.04, 0.04 |
| | Ave.= 1.063 | Ave. = 0.04 |
| | Std. Dev. = 0.057 | Std. Dev. = 0 |
| 432 | 1.003, 1.082 | 0.1, 0.1 |
| | Ave. = 1.0425 | Ave. = 0.1 |
| | Std. Dev. = 0.056 | Std. Dev. = 0 |
| 768 | 1.011, 1.093 | 1.28, 1.26 |
| | Ave. = 1.052 | Ave. = 1.27 |
| | Std. Dev. = 0.058 | Std. Dev. = 0.01 |

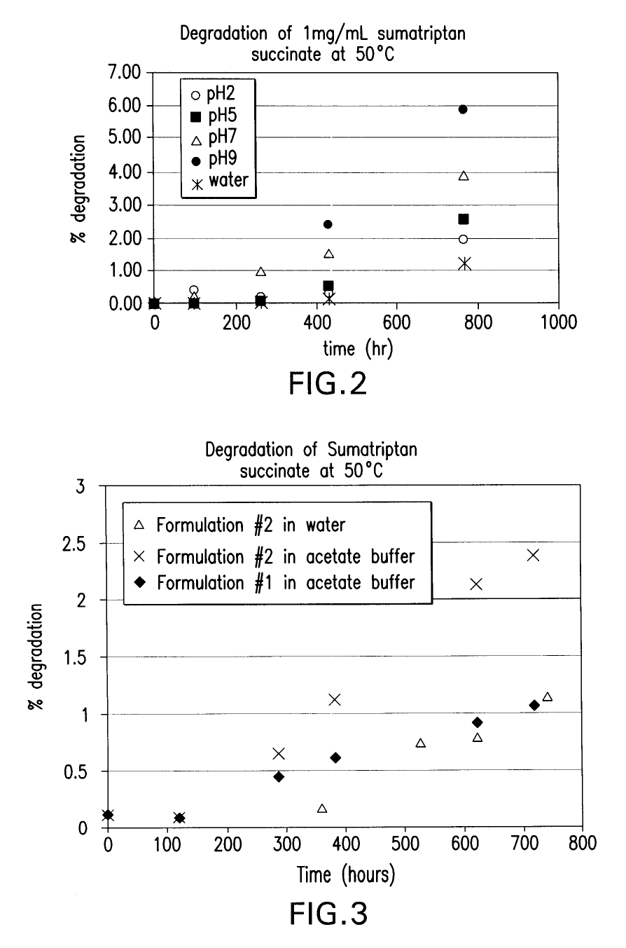

FIG. 2 illustrates the degradation of sumatriptan in mg/mL at different pHs as a function of time. The data presented in FIG. 2 is an average of three individual sample tubes. As used herein, degradation in percent area by area is that amount of degradation that is the ratio of the sum total of degradation peaks divided by the response factor (sumatriptan's standard curve slope value) multiplied by 100, and not a quantified amount of degradation. Therefore, it is possible that degradation is higher than the values shown, due to lower response factors for degradation products. This percent, however, was used in a relative comparison of sumatriptan's stability in various pHs.

The data indicates that sumatriptan has the greatest stability in water, followed by buffers at a pH of 2, 5, 7, and finally 9. Based on the trend illustrated in FIG. 2, water as well as a low pH buffer were considered to be the best choices for oral sumatriptan composition development. An oral solution, however, at a pH of 2 is generally not tolerable and therefore, a pH 5 acetate buffer was considered for further formulation development. This decision was further supported by the fact that the marketed nasal spray is formulated at a pH of 5. Also, additional studies (not presented here) demonstrated that sumatriptan was very stable in buffers with a pH of about 2 to 5, with only 1.5 percent degradation after one month at 50° C.

### Example 2.2

### Effect of Various Excipients on Stability

To determine the most compatible excipients to be used in an oral composition for sumatriptan, the following screening techniques were developed.

A) Stage I: Solubilizers

Sumatriptan succinate was used to make solutions in an acetate buffer at a pH of 5. Solutions were made in 34 mL Pyrex® brand borosilicate glass tubes. Twenty mg of sumatriptan succinate was weighed and dissolved in 10 mL of a 0.1 M acetate buffer at a pH of 5. To this solution was added 10 mL of various solubilizers either in solution or aqueous solution. These mixtures were thoroughly mixed and a 1 mL aliquot removed for HPLC analysis. The final concentration of sumatriptan in the solutions after mixing with various solubilizers was 1 mg/mL. The solubilizers tested were polyethylene glycol 400 (PEG 400), polysorbate 80 or Tween™ 80, polyvinylpyrrolidone K30 (PVP K30), glycerin, hydroxypropylmethylcellulose (HPMC), and xanthan gum. Hydroxypropylmethylcellulose and xanthan gum are typically used in syrup and suspension formulations. These solutions were then incubated for 48 hours at 50° C. to determine if the sumatriptan would remain in solution and if any excipients demonstrated visible immiscibility in the buffer. The tubes were then placed in a 50° C. oven until the next stage of the excipient screening process.

The results of this initial screen are provided in Table 7 below. The percent of sumatriptan recovered, i.e., the experimentally determined amount (mg/mL) of sumatriptan divided by the theoretical amount present at each stage, is also provided. These percentages are often greater than 100 percent because of experimental variation when weighing sumatriptan or the excipients and splitting the samples. These percentages should therefore be interpreted as merely an indication that the combination of these excipients does not lead to precipitation of sumatriptan out of solution.

TABLE 7

| | Percent | Percent of |
|---|---|---|
| | Degradation | Sumatriptan |
| | After 1 month at | Recovered |
| Solubilizer | 50° C. | in Solution |
| PEG 400 | 14 | 60 |
| Tween ™ 80 | 0.49 | 106 |
| PVP K30 | 0.37 | 102 |
| Glycerin | 0.12 | 115 |
| HPMC | 0.64 | 98 |
| Xanthan Gum | 0.07 | 106 |

Based on the results, PEG 400, Tween™ 80, and HPMC had higher percent degradation, with PEG 400 demonstrating the greatest percent degradation. The percent of sumatriptan recovered in solution for PEG 400 was also markedly lower compared to the other solubilizers. In contrast, PVP K30, glycerin, and xanthan gum had lower percent degradation, with xanthan gum surprisingly demonstrating the lowest percent degradation even in solution form. The percent of sumatriptan determined in solution for these excipients was high, demonstrating that their presence does not lead to precipitation of sumatriptan.

B) Stage II: Sweeteners

The second stage of the excipient screen involved a number of typical sweeteners used in oral solutions. Each of the original sample tubes from Stage I was split, some of the original mixture was left as a control, and the rest was used to mix with various sweeteners. In particular, after 48 hours, each sample tube from Stage I was divided into three new glass tubes with 5 mL aliquots of the original mixture and the remaining 4 mL kept as a control. To these aliquots were added 5 mL of either sorbitol, saccharin (0.5 percent (w/v) aqueous solution), or sucrose solution (liquid sugar). The concentration of sumatriptan in these samples was reduced to 0.5 mg/mL. These samples were then placed into a 50° C. oven for 48 hours to determine if any of the excipients demonstrated visible immiscibility or turbidity. The results of this screening stage are provided in Table 8 below.

TABLE 8

| | Percent | Percent of |
|---|---|---|
| | Degradation | Sumatriptan |
| | After 1 month | Recovered |
| Solubilizer + Sweetener | at 50° C. | in Solution |
| PEG 400 + Sorbitol | 3 | 80.4 |
| PEG 400 + Saccharin | 13 | 52.8 |
| PEG 400 + Sucrose | 7 | 73.8 |
| Tween ™ 80 + Sorbitol | 0 | 113 |
| Tween ™ 80 + Saccharin | 1.03 | 101 |
| Tween ™ 80 + Sucrose | 5.29 | 61 |
| PVP K30 + Sorbitol | 0.04 | 113 |
| PVP K30 + Saccharin | 0.17 | 81 |
| PVP K30 + Sucrose | 0.31 | 103 |
| Glycerin + Sorbitol | 0 | 119 |
| Glycerin + Saccharin | 0.15 | 117 |
| Glycerin + Sucrose | 0.06 | 133 |
| HPMC + Saccharin | 0.1 | 117 |
| Xanthan Gum + Sorbitol | 0 | 111 |
| Xanthan Gum + Saccharin | 0.03 | 140 |
| Xanthan Gum + Sucrose | 0.27 | 111 |

Consistent with the results in Stage I, PEG 400 demonstrated the least compatibility with sumatriptan by showing significantly higher percent degradations compared to PVP K30, glycerin, and xanthan gum. Once again, the percent of sumatriptan recovered in solution for PEG 400 was significantly lower compared to the other solubilizers. Glycerin, PVP K30, HPMC and xanthan gum all had percent degradation values below 0.4 percent, with the highest percent degradation of 0.31 percent recorded for PVP K30 and sugar. Surprisingly, sorbitol reduced the degradation of several excipients to zero (0) or close to zero (0) even when combined with excipients such as TweenTm and PVP that showed some degradation as shown in Table 8 above. The most stable solutions were combinations of Tween™ 80 and sorbitol, glycerin and sorbitol, and xanthan gum and sorbitol.

The stability and solubility of sumatriptan compositions in acetate buffer with just the sweeteners was also tested. These samples were mixed in a 1:1 ratio. The results of this study are provided in Table 9 below.

TABLE 9

| | Percent Degradation After 1 | Percent of Sumatriptan |
|---|---|---|
| Sweetener | month at 50° C. | Recovered in Solution |
| Sucralose | 0.44 | 99 |
| Liquid Sugar | 0.54 | 111 |
| Saccharin | 0.04 | 114 |
| Sorbitol | 0 | 124 |

Saccharin and sorbitol exhibited lower percent degradation compared to sucralose and liquid sugar. Saccharin and sorbitol also maintained a higher percentage of sumatriptan in solution.

C) Stage III: Preservatives

The final stage of the excipient screen was used to establish a compatible preservative. From each of the Stage II samples (solubilizer with sweeteners), some of the sample was left as a control and the remaining volume used to mix with either sodium benzoate or methyl paraben. Specifically, Stage II samples were removed and split into two tubes each containing 2.5 mL and the remaining solution kept as a control. To the 2.5 mL aliquot was added 2.5 mL of a 1 percent (w/v) sodium benzoate aqueous solution, benzyl alcohol (in liquid form), or a 0.2 percent (w/v) methyl paraben aqueous solution. These solutions were then stored at 50° C. for one month and periodically analyzed by HPLC. The results of these studies are provided in Table 10 below.

TABLE 10

| | Percent | Percent of |
|---|---|---|
| | Degradation | Sumatriptan |
| Solubilizer + Sweetener + | After 1 month | Recovered |
| Preservative | at 50° C. | in Solution |
| Tween ™ 80 + Sorbitol + | 8.61 | 84 |
| Sodium Benzoate | | |
| Tween ™ 80 + Sorbitol + | 0.25 | 113 |
| Methyl Paraben | | |
| Tween ™ 80 + Saccharin + | 8.72 | 64 |
| Sodium Benzoate | | |
| Tween ™ 80 + Saccharin + | 1.32 | 91 |
| Methyl Paraben | | |
| Tween ™ 80 + Sugar + | 8.27 | 87 |
| Sodium Benzoate | | |
| Tween ™ 80 + Sugar + | 3.43 | 93 |
| Methyl Paraben | | |
| PVP K30 + Sorbitol + | 0.27 | 117 |
| Sodium Benzoate | | |
| PVP K30 + Sorbitol + Methyl | 0.25 | 113 |
| Paraben | | |
| PVP K30 + Saccharin + | 0.37 | 109 |
| Sodium Benzoate | | |
| PVP K30 + Saccharin + | 0.3 | 119 |
| Methyl Paraben | | |
| PVP K30 + Sugar + Sodium | 1.28 | 94 |
| Benzoate | | |
| PVP K30 + Sugar + Methyl | 0.37 | 98 |
| Paraben | | |
| Glycerin + Sorbitol + Sodium | 0.93 | 130 |
| Benzoate | | |
| Glycerin + Sorbitol + Methyl | 0.3 | 120 |
| Paraben | | |
| Glycerin + Saccharin + | 0.24 | 116 |
| Sodium Benzoate | | |
| Glycerin + Saccharin + | 0.34 | 119 |
| Methyl Paraben | | |
| Glycerin + Sugar + Sodium | 0.55 | 146 |
| Benzoate | | |
| Glycerin + Sugar + Methyl | 0.38 | 85 |
| Paraben | | |
| HPMC + Saccharin + Sodium | 0.27 | 117 |
| Benzoate | | |
| HPMC + Saccharin + Methyl | 0.68 | 118 |
| Paraben | | |
| Xanthan Gum + Sorbitol + | 0.38 | 106 |
| Sodium Benzoate | | |
| Xanthan Gum + Sorbitol + | 0.34 | 116 |
| Methyl Paraben | | |
| Xanthan Gum + Saccharin + | 0.27 | 105 |
| Sodium Benzoate | | |
| Xanthan Gum + Saccharin + | 0.12 | 103 |
| Methyl Paraben | | |
| Xanthan Gum + Sugar + | 1.53 | 104 |
| Sodium Benzoate | | |
| Xanthan Gum + Sugar + | 1.07 | 75 |
| Methyl Paraben | | |

Generally, sumatriptan compositions with sodium benzoate had higher percent degradations compared to compositions with methyl paraben. Compositions that included Tween™ 80 with sodium benzoate had the highest percent degradation, with values over 8 percent. In contrast, compositions that included PVP K30, glycerin, HPMC, and xanthan gum had percent degradation values less than 2 percent, with the highest percent degradation of 1.53 percent recorded for xanthan gum, sugar, and sodium benzoate. Again, one of the most stable solutions included Tween™, sorbitol and methyl paraben. PVP with either sweetener or preservative was generally as stable, but only xanthan gum, saccharin, and methyl paraben was substantially lower.

The stability and solubility of sumatriptan compositions in acetate buffer with the preservative was also tested. These samples were mixed in a 1:1 ratio. The results of this study are provided in Table 11 below.

TABLE 11

| | Percent Degradation After 1 | Percent of Sumatriptan |
|---|---|---|
| Preservative | month at 50° C. | Recovered in Solution |
| Sodium Benzoate | 0.17 | 114 |
| Methyl Paraben | 0.24 | 107 |

D) Observations and Conclusions

Based on the data above, it can be seen that PEG 400 and Tween™ 80 (except when in solution with sorbitol) demonstrated the least compatability with sumatriptan. One characteristic that distinguished these samples was a yellow color change that occurred under accelerated stability conditions at 50° C. for any combination of the PEG 400 or Tween™ 80 with sumatriptan and liquid sugar. In contrast, sorbitol, saccharin, PVP K30 and glycerin were the most compatible excipients for sumatriptan solutions. Xanthan gum and HPMC are traditionally used in syrups or suspensions.

### Example 3

### Oral Liquid Sumatriptan Compositions According to the Invention

Concurrently with the excipient screen, a series of prototype formulations were prepared to evaluate the combination of excipients that demonstrated compatibility with sumatriptan. The solubility and stability of sumatriptan in these formulations were studied at both room temperature and at 50° C. for one month. Formulations 1 and 2 were prepared in water or a pH 5 acetate buffer to determine if the buffer would stabilize sumatriptan succinate compared to water. The stability of these formulations in plastic was also studied to determine potential instability or adsorption with polyethylene bottles. Details regarding Formulations 1 and 2 are provided below.

Formulation 1

| | | |
|---|---|---|
| | | Amount |
| Ingredient | Amount | Per 20 mL |

| | | | | |
|---|---|---|---|---|
| Liquid Sugar | 40% | v/v | 8 | mL |
| Acetate Buffer at pH of 5 or | 59.5% | v/v | 11.9 | mL |
| Water | | | | |
| Sumatriptan Succinate | 12.5 | mg/mL | 250 | mg |
| Sodium Benzoate | 0.01 | mg/mL | 0.2 | mg |
| Tween ™ 80 | 0.5% | v/v | 0.1 | mL |

For Formulation 1, the order of addition surprisingly and unexpectedly influenced the ability of sumatriptan to dissolve in solution. If sumatriptan was added after all the other components, the solution appeared very cloudy. When sumatriptan was dissolved in water first, however, then liquid sugar added slowly, followed by the other excipients, the solution was surprisingly clear in spite of the cloudy result from the different mixing order. Based on this observation, the solubility and stability of Formulation 1 in water was studied at ambient temperature and was not studied at 50° C. A study of Formulation 1 in acetate buffer, however, was studied at 50° C. When a comparison of Formulation 1 at ambient temperature in water was made with Formulation 1 in acetate buffer, the stability of sumatriptan was found to be superior in the acetate buffer formulation. At the specified time, samples from each formulation were removed using a micropipette and aliquoted into a known volume of milliQ water to give the desired dilution. The results of these stability studies at ambient temperature are provided in Tables 12 and 13, and at 50° C. in Table 14. FIG. 3 show the results graphically. Therefore, Formulation 1 in acetate buffer was further studied under accelerated conditions.

TABLE 12
(Ambient Temperature)

| | Sumatriptan | | |
|---|---|---|---|
| | Amount in | | |
| | Formulation | Degradation | |
| Time | 1 in Water | (Percent | Percent |
| (Hours) | (mg/mL) | Area by Area) | Degradation |
| 24 | 11.6098 | — | — |
| 120 | 8.422 | 0.0098 | 0.1164 |
| 288 | 11.946 | 0.0116 | 0.0971 |
| 456 | 12.238 | 0.0261 | 0.2133 |
| 552 | 12.096 | 0.016 | 0.1323 |
| 792 | 9.64 | 0.0751 | 0.779 |

Table 12 illustrates preliminary data that is useful for identifying degradation/stability trends relative to other formulations.

TABLE 13
(Ambient Temperature)

| | Sumatriptan Amount in | | Percent |
|---|---|---|---|
| Time | Formulation 1 in | Degradation (Percent | Degra- |
| (Hours) | Acetate Buffer (mg/mL) | Area by Area) | dation |
| 0 | 17.040, 17.226, 17.091 | 0.0184, 0.0197, 0.0195 | 0.1121 |
| | Ave. = 17.119 | Ave. = 0.0192 | |
| | Std. Dev. = 0.096 | Std. Dev. = 0.0007 | |
| 120 | 12.258, 11.933, 11.896 | 0.0102, 0.0095, 0.0090 | 0.0795 |
| | Ave. = 12.029 | Ave. = 0.0096 | |
| | Std. Dev. = 0.199 | Std. Dev. = 0.0006 | |
| 288 | 11.344, 11.233, 11.231 | 0.0147, 0.0303, 0.0210 | 0.1952 |
| | Ave. = 11.269 | Ave. = 0.0220 | |
| | Std. Dev. = 0.065 | Std. Dev. = 0.0078 | |
| 384 | 11.984, 12.726, 12.211 | 0.0211, 0.0250, 0.0158 | 0.1677 |
| | Ave. = 12.307 | Ave. = 0.0206 | |
| | Std. Dev. = 0.380 | Std. Dev. = 0.0046 | |
| 624 | 11.572, 11.61, 12.244 | 0.0503, 0.0576, 0.0387 | 0.4138 |
| | Ave. = 11.809 | Ave. = 0.0489 | |
| | Std. Dev. = 0.377 | Std. Dev. = 0.0095 | |
| 720 | 10.013, 7.974, 8.795 | 0.0603, 0.0449, 0.0232 | 0.4794 |
| | Average = 8.927 | Ave. = 0.0428 | |
| | Standard | Std. Dev. = 0.0186 | |
| | Deviation = 1.026 | | |

TABLE 14
(50° C.)

| | Sumatriptan Amount in | | Percent |
|---|---|---|---|
| Time | Formulation 1 | Degradation (Percent | Degra- |
| (Hours) | Acetate Buffer (mg/mL) | Area by Area) | dation |
| 0 | 17.040, 17.226, 17.091 | 0.0184, 0.0197, 0.0195 | 0.1121 |
| | Ave. = 17.119 | Ave. = 0.0192 | |
| | Std. Dev. = 0.096 | Std. Dev. = 0.0007 | |
| 120 | 12.062, 11.015, 11.887 | 0.0104, 0.0105, 0.0100 | 0.0884 |
| | Ave. = 11.655 | Ave. = 0.0103 | |
| | Std. Dev. = 0.561 | Std. Dev. = 0.0003 | |
| 288 | 12.632, 10.845, 11.015 | 0.0623, 0.0500, 0.0399 | 0.4413 |
| | Ave. = 11.497 | Ave. = 0.0507 | |
| | Std. Dev. = 0.986 | Std. Dev. = 0.0112 | |
| 384 | 12.61, 11.939, 11.893 | 0.0738, 0.0818, 0.0657 | 0.6073 |
| | Ave. = 12.147 | Ave. = 0.0738 | |
| | Std. Dev. = 0.401 | Std. Dev. = 0.0081 | |
| 624 | 11.465, 11.292, 11.164 | 0.106, 0.1098, 0.0939 | 0.913 |
| | Ave. = 11.307 | Ave. = 0.1032 | |
| | Std. Dev. = 0.151 | Std. Dev. = 0.0083 | |
| 720 | 10.362, 10.806, 10.248 | 0.1032, 0.1254, 0.1021 | 1.0526 |
| | Ave. = 10.472 | Ave. = 0.1102 | |
| | Std. Dev. = 0.295 | Std. Dev. = 0.00131 | |

Formulation 2 was designed as a sugar-free solution in which sucralose was substituted for liquid sugar. In this formulation, HPMC K4 was used to increase solution viscosity.

Formulation 2

| | | |
|---|---|---|
| | | Amount |
| Ingredient | Amount | Per 20 mL |

| | | | | |
|---|---|---|---|---|
| Sucralose | 0.1 | mg/mL | 2 | mg |
| Acetate buffer at | 94.5% | v/v | 18.9 | mL |
| pH of 5 or Water | | | | |
| Sumatriptan | 12.5 | mg/mL | 250 | mg |
| Succinate | | | | |
| Sodium Benzoate | 0.01 | mg/mL | 0.2 | mg |
| Tween ™80 | 0.5% | v/v | 0.1 | mL |
| HPMC K4 | 0.1 | mg/mL | 2 | mg |

The results of the stability studies for Formulation 2 at ambient temperature are provided in Tables 15 and 16, and at 50° C. in Tables 17 and 18.

TABLE 15
(Ambient Temperature)

| | Sumatriptan Amount in | | Percent |
|---|---|---|---|
| Time | Formulation 2 in Water | Degradation | Degra- |
| (Hours) | (mg/mL) | (mg/mL) | dation |
| 0 | 8.938, 9.094, 8.703 | 0, 0, 0 | 0 |
| | Ave. = 8.912 | Ave. = 0 | |
| | Std. Dev. = 0.197 | Std. Dev. = 0 | |
| 96 | 11.951, 11.956, 11.804 | 0, 0, 0 | 0 |
| | Ave. = 11.904 | Ave. = 0 | |
| | Std. Dev. = 0.086 | Std. Dev. = 0 | |
| 192 | 8.624, 8.639, 8.541 | 0.012, 0.0124, 0.0121 | 0.141 |
| | Ave. = 8.601 | Ave. = 0.0122 | |
| | Std. Dev. = 0.053 | Std. Dev. = 0.0002 | |
| 360 | 10.595, 10.864, 11.469 | 0.0529, 0.0506, | 0.565 |
| | Ave. = 10.976 | 0.0826 | |
| | Std. Dev. = 0.448 | Ave. = 0.062 | |
| | | Std. Dev. = 0.0178 | |
| 478 | 12.280, 12.184, 12.101 | 0.0274, 0.0304, | 0.236 |
| | Ave. = 12.188 | 0.0286 | |
| | Std. Dev. = 0.090 | Ave. = 0.0288 | |
| | | Std. Dev. = 0.0015 | |
| 574 | 12.856, 12.652, 12.557 | 0.027, 0.0298, 0.0272 | 0.221 |
| | Ave. = 12.688 | Ave. = 0.028 | |
| | Std. Dev. = 0.153 | Std. Dev. = 0.0016 | |
| 792 | 12.061, 12.219, 12.472 | 0.0336, 0.0413, | 0.29 |
| | Ave. = 12.251 | 0.0317 | |
| | Std. Dev. = 0.207 | Ave. = 0.0355 | |
| | | Std. Dev. = 0.0051 | |

TABLE 16
(Ambient Temperature)

| | Sumatriptan Amount in | | Percent |
|---|---|---|---|
| Time | Formulation 2 in | Degradation | Degra- |
| (Hours) | Acetate Buffer (mg/mL) | (mg/mL) | dation |
| 0 | 16.868, 17.155, 17.030 | 0.0182, 0.0192, | 0.111 |
| | Ave. = 17.018 | 0.0194 | |
| | Std. Dev. = 0.144 | Ave. = 0.0189 | |
| | | Std. Dev. = 0.0006 | |
| 120 | 11.400, 11.449, 11.419 | 0.0088, 0.0094, | 0.084 |
| | Ave. = 11.423 | 0.0105 | |
| | Std. Dev. = 0.025 | Ave. = 0.0096 | |
| | | Std. Dev. = 0.0009 | |
| 288 | 11.757, 12.156, 11.773 | 0.0143, 0.0117, | 0.119 |
| | Ave. = 11.895 | 0.0163 | |
| | Std. Dev. = 0.0226 | Ave. = 0.0141 | |
| | | Std. Dev. = 0.0023 | |
| 384 | 11.757, 12.156, 11.773 | 0.0227, 0.0132, 0.02 | 0.151 |
| | Ave. = 11.895 | Ave. = 0.0186 | |
| | Std. Dev. = 0.0226 | Std. Dev. = 0.0049 | |
| 624 | 11.99, 11.457, 12.632 | 0.0386, 0.0273, | 0.312 |
| | Ave. = 12.026 | 0.0468 | |
| | Std. Dev. = 0.588 | Ave. = 0.0376 | |
| | | Std. Dev. = 0.0098 | |
| 720 | 10.337, 11.131, 12.672 | 0.0332, 0.0268, | 0.32 |
| | Ave. = 11.380 | 0.0494 | |
| | Std. Dev. = 1.187 | Ave. = 0.0365 | |
| | | Std. Dev. = 0.0116 | |

TABLE 17
(50° C.)

| | Sumatriptan Amount in | | Percent |
|---|---|---|---|
| Time | Formulation 2 in Water | Degradation | Degra- |
| (Hours) | (mg/mL) | (mg/mL) | dation |
| 360 | 12.202, 12.179, 12.096 | 0.0197, 0.02, 0.0225 | 0.171 |
| | Ave. = 12.159 | Ave. = 0.021 | |
| | Std. Dev. = 0.056 | Std. Dev. = 0.002 | |
| 528 | 11.593, 11.785, 11.785 | 0.0923, 0.0867, | 0.737 |
| | Ave. = 11.721 | 0.0803 | |
| | Std. Dev. = 0.111 | Ave. = 0.086 | |
| | | Std. Dev. = 0.006 | |
| 624 | 13.157, 12.610, 11.542 | 0.1055, 0.0948, | 0.781 |
| | Ave. = 12.436 | 0.0909 | |
| | Std. Dev. = 0.821 | Ave. = 0.097 | |
| | | Std. Dev. = 0.008 | |
| 742 | 10.931, 12.712, 11.204 | 0.1241, 0.1478, | 1.132 |
| | Ave. = 11.616 | 0.1225 | |
| | Std. Dev. = 0.959 | Ave. = 0.131 | |
| | | Std. Dev. = 0.014 | |

TABLE 18
(50° C.)

| | Sumatriptan Amount in | | Percent |
|---|---|---|---|
| Time | Formulation 2 in | Degradation | Degra- |
| (Hours) | Acetate Buffer (mg/mL) | (mg/mL) | dation |
| 0 | 16.868, 17.155, 17.030 | 0.0182, 0.0192, | 0.111 |
| | Ave. = 17.018 | 0.0194 | |
| | Std. Dev. = 0.144 | Ave. = 0.0189 | |
| | | Std. Dev. = 0.0006 | |
| 120 | 12.171, 11.967, 11.554 | 0.0096, 0.0104, | 0.083 |
| | Ave. = 11.897 | 0.0097 | |
| | Std. Dev. = 0.314 | Ave. = 0.0099 | |
| | | Std. Dev. = 0.0004 | |
| 288 | 10.984, 11.273, 11.123 | 0.0777, 0.0722, | 0.653 |
| | Ave. = 11.127 | 0.0680 | |
| | Std. Dev. = 0.145 | Ave. = 0.0726 | |
| | | Std. Dev. = 0.0049 | |
| 384 | 11.547, 10.47, 11.668 | 0.1309, 0.1203, | 1.111 |
| | Ave. = 11.228 | 0.1231 | |
| | Std. Dev. = 0.660 | Ave. = 0.1248 | |
| | | Std. Dev. = 0.0055 | |
| 624 | 10.104, 10.537, 10.662 | 0.2001, 0.2488, | 2.13 |
| | Ave. = 10.434 | 0.2178 | |
| | Std. Dev. = 0.293 | Ave. = 0.2222 | |
| | | Std. Dev. = 0.0247 | |
| 720 | 10.462, 10.682, 10.316 | 0.2338, 0.2809, | 2.383 |
| | Ave. = 10.487 | 0.2351 | |
| | Std. Dev. = 0.184 | Ave. = 0.2499 | |
| | | Std. Dev. = 0.0268 | |

The stability of Formulation 2 in acetate buffer or water at ambient conditions was improved compared to Formulation 1. However, under accelerated stability studies conducted at 50° C. Formulation 2 in water and Formulation 1 in acetate buffer had similar stability. This is graphically illustrated in FIG. 3. Also, both formulations underwent a yellow color change which was not observed in their controls. Formulation 2 in acetate buffer, however, was much less stable than in water. Interestingly, there was no significant difference in the pH of Formulation 2 after one month at 50° C. depending on whether acetate buffer or water was used. This surprisingly and unexpectedly demonstrated that the presence of the buffer does not increase the stability of Formulation 2.

Other formulations having slight variations were tested to determine the optimal combination of excipients. For example, Formulation 3 and 3a are the same with the exception of the preservative used. Formulation 3 uses sodium benzoate, while Formulation 3a uses methyl paraben.

Formulation 3

| | | |
|---|---|---|
| | | Amount |
| Ingredient | Amount | Per 20 mL |

| | | | | |
|---|---|---|---|---|
| Sorbitol | 20% | v/v | 4 | mL |
| Water | 68.5% | v/v | 13.7 | mL |
| Sumatriptan Succinate | 12.5 | mg/mL | 250 | mg |
| Sodium Benzoate | 0.01 | mg/mL | 0.2 | mg |
| Tween ™80 | 0.5% | v/v | 0.1 | mL |
| PVP K30 | 0.1 | mg/mL | 2.0 | mg |

Formulation 3a

| | | |
|---|---|---|
| | | Amount |
| Ingredient | Amount | Per 100 mL |

| | | | | |
|---|---|---|---|---|
| Sorbitol | 20% | v/v | 20 | mL |
| Water | 68.5% | v/v | 68.5 | mL |
| Sumatriptan Succinate | 10 | mg/mL | 1 | g |
| Methyl Paraben | 0.05 | mg/mL | 5.0 | mg |
| Tween ™ 80 | 0.5% | v/v | 0.5 | mL |
| PVP K30 | 0.1 | mg/mL | 10.0 | mg |

Compared to Formulation 3, Formulation 4 contains glycerin instead of sorbitol and includes potassium sorbate as a preservative.

Formulation 4

| | | |
|---|---|---|
| | | Amount |
| Ingredient | Amount | Per 20 mL |

| | | | | |
|---|---|---|---|---|
| Acetate Buffer at pH of 5 | 84% | v/v | 16.8 | mL |
| Sumatriptan Succinate | 5 | mg/mL | 100 | mg |
| PVP K30 | 0.05 | mg/mL | 1 | mg |
| Glycerin | 10% | v/v | 2 | mL |
| Sodium Saccharin | 0.02 | mg/mL | 0.4 | mg |
| Potassium Sorbate | 0.01 | mg/mL | 0.2 | mg |
| Tween ™ 80 | 0.5% | v/v | 0.1 | mL |

Formulation 5 is similar to Formulation 3a with the addition of sucralose.

Formulation 5

| | | |
|---|---|---|
| | | Amount |
| Ingredient | Amount | Per 10 mL |

| | | | | |
|---|---|---|---|---|
| Sorbitol | 20% | v/v | 2 | mL |
| Water | 63.5% | v/v | 6.35 | mL |
| Sumatriptan Succinate | 5 | mg/mL | 50 | mg |
| Methyl Paraben | 0.1 | mg/mL | 1 | mg |
| Sucralose | 0.05 | mg/mL | 0.5 | mg |
| Tween ™ 80 | 0.5% | v/v | 0.05 | mL |
| PVP K30 | 0.1 | mg/mL | 1 | mg |

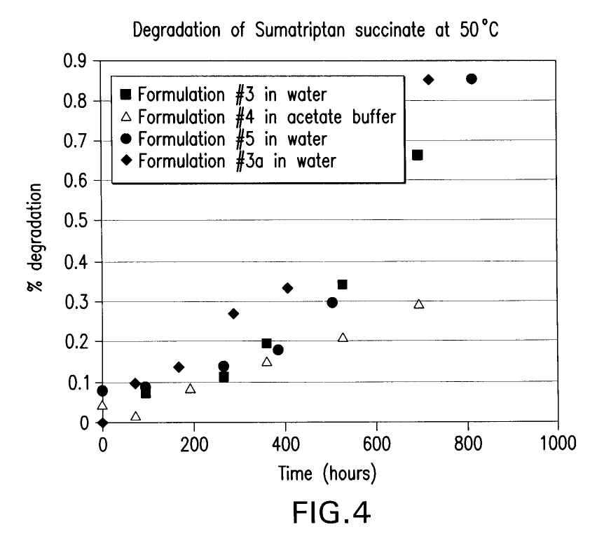

The stability of these formulations was studied for one month at 50° C., with results presented below in Tables 19-22, and shown in FIG. 4. For each time point shown in FIG. 4, samples from each formulation were removed using a micropipette and aliquoted into known volume of milliQ water to give the desired dilution.

TABLE 19
(50° C.)

| | | | Percent |
|---|---|---|---|
| Time | Sumatriptan Amount in | Degradation (Percent | Degra- |
| (Hours) | Formulation 3 (mg/mL) | Area by Area) | dation |
| 96 | 11.614 | 0.0087 | 0.0749 |
| 264 | 13.759 | 0.0157 | 0.1141 |
| 360 | 13.759 | 0.0269 | 0.1955 |
| 528 | 13.068 | 0.0446 | 0.3413 |
| 696 | 13.282 | 0.0883 | 0.6648 |

TABLE 20
(50° C.)

| | | | Percent |
|---|---|---|---|
| Time | Sumatriptan Amount in | Degradation (Percent | Degra- |
| (Hours) | Formulation 3a (mg/mL) | Area by Area) | dation |
| 0 | 9.89 | 0 | 0 |
| 72 | 11.859 | 0.0115 | 0.097 |
| 168 | 10.367 | 0.0142 | 0.137 |
| 288 | 9.636 | 0.026 | 0.27 |
| 408 | 9.385 | 0.0312 | 0.332 |
| 720 | 8.495 | 0.0723 | 0.851 |

TABLE 21
(50° C.)

| | | | Percent |
|---|---|---|---|
| Time | Sumatriptan Amount in | Degradation (Percent | Degra- |
| (Hours) | Formulation 4 (mg/mL) | Area by Area) | dation |
| 0 | 5.454, 5.441, 5.373 | 0.0018, 0.0035, 0.0022 | 0.0461 |
| | Ave. = 5.4227 | Ave. = 0.0025 | |
| | Std. Dev. = 0.0435 | Std. Dev. = 0.0009 | |
| 72 | 5.1156, 5.1043, 5.1983 | 0.001, 0.0006, 0.001 | 0.0169 |
| | Ave. = 5.1394 | Ave. = 0.0009 | |
| | Std. Dev. = 0.0513 | Std. Dev. = 0.0002 | |
| 192 | 4.968, 4.95, 4.873 | 0.0043, 0.004, 0.0043 | 0.0852 |
| | Ave. = 4.9303 | Ave. = 0.0042 | |
| | Std. Dev. = 0.0505 | Std. Dev. = 0.0002 | |
| 360 | 5.211, 4.935, 4.915 | 0.0075, 0.0081, 0.0072 | 0.1514 |
| | Ave. = 5.0203 | Ave. = 0.0076 | |
| | Std. Dev. = 0.1654 | Std. Dev. = 0.0005 | |
| 528 | 4.93, 5.073, 5.009 | 0.011, 0.0106, 0.0101 | 0.2112 |
| | Ave. = 5.004 | Ave. = 0.0106 | |
| | Std. Dev. = 0.0716 | Std. Dev. = 0.0005 | |
| 696 | 4.94, 4.93, 4.901 | 0.0147, 0.0147, 0.014 | 0.2938 |
| | Ave. = 4.9237 | Ave. = 0.0145 | |
| | Std. Dev. = 0.0203 | Std. Dev. = 0.0004 | |

TABLE 22
(50° C.)

| | | | Percent |
|---|---|---|---|
| Time | Sumatriptan Amount in | Degradation (Percent | Degra- |
| (Hours) | Formulation 5 (mg/mL) | Area by Area) | dation |
| 0 | 5.274, 5.389 | 0.0042, 0.0044 | 0.0807 |
| | Ave. = 5.332 | Ave. = 0.0043 | |
| | Std. Dev. = 0.0813 | Std. Dev. = 0.0001 | |
| 96 | 5.188, 5.338 | 0.0046, 0.0048 | 0.0893 |
| | Ave. = 5.263 | Ave. = 0.0047 | |
| | Std. Dev. = 0.1061 | Std. Dev. = 0.0001 | |
| 264 | 5.494 | 0.0076 | 0.1383 |
| | Ave. = 5.494 | Ave. = 0.0076 | |
| 384 | 5.328, 5.31 | 0.0009, 0.01 | 0.1786 |
| | Ave. = 5.319 | Ave. = 0.0095 | |
| | Std. Dev. = 0.0127 | Std. Dev. = 0.0007 | |
| 504 | 5.191, 5.069 | 0.0014, 0.01163 | 0.2953 |
| | Ave. = 5.130 | Ave. = 0.0152 | |
| | Std. Dev. = 0.0863 | Std. Dev. = 0.0016 | |
| 816 | 4.827, 4.803 | 0.0395, 0.0427 | 0.8536 |
| | Ave. = 4.815 | Ave. = 0.0411 | |
| | Std. Dev. = 0.0170 | Std. Dev. = 0.0023 | |

Similar to Formulations 1 and 2, Formulations 5 and 3a also underwent a light yellow color change after one month at 50° C., while their controls remained colorless. Formulations 3a and 4 did not undergo as large a color change, however, which suggested that sucralose, liquid sugar or sodium benzoate may not be as compatible with sumatriptan in Formulations 1, 2, and 5.

The percent degradation for one month stability studies of these formulations is presented in FIG. 4. This percentage was calculated as previously described. When comparing Formulations 1 through 5, the formulation with the least degradation was Formulation 4, which was used as the basis for further formulation development.

The concentration of sumatriptan and PVP in Formulation 4 was not within the preferred range, however, e.g., 10 mg/mL to 50 mg/mL of sumatriptan base and did not encompass an exemplary amount of 25 mg/mL sumatriptan. Therefore, other formulations were created to be within the expected dosage for both the sumatriptan and having a suitable amount of PVP excipient. The amount of PVP was increased significantly to 4.4 percent (w/v) from 0.005 percent (w/v) in Formulation 4 to utilize the taste masking characteristics of PVP. Because PVP K30 has a Center for Drug Evaluation and Research (CDER) listed maximal level at 3 percent (w/v) in oral solutions, however, PVP K25 was used instead. This type of PVP is listed with the CDER as having an approved usage of up to 50 percent (w/v) in oral solution.

Formulations 6 and 6a (described below) are relatively similar to Formulation 4 except water was used instead of acetate buffer. Also, Tween™ 80 was not included in either Formulations 6 or 6a based on data from the excipient screen which demonstrated that Tween™ 80 in a binary or tertiary combination with sumatriptan was not stable. In Formulation 6a, glycerin was used in place of PVP. The reason for this change was that sumatriptan was found to be very stable when in combination with sorbitol and glycerin based on data from the excipient screen. Additionally, production of the formulation if suitable would be less costly and time-consuming since PVP would not need to be weighed out and dissolved.

Formulation 6

| | | |
|---|---|---|
| | | Amount |
| Ingredient | Amount | Per 25 mL |

| | | | | |
|---|---|---|---|---|
| Sorbitol | 20% | v/v | 5 | mL |
| Water | 80% | v/v | 20 | mL |
| Sumatriptan Succinate | 25 | mg/mL | 625 | mg |
| PVP K25 | 44 | mg/mL | 1.1 | g |
| Potassium Sorbate | 1 | mg/mL | 25 | mg |

Formulation 6a

| | | |
|---|---|---|
| | | Amount |
| Ingredient | Amount | Per 25 mL |

| | | | | |
|---|---|---|---|---|
| Sorbitol | 10% | v/v | 2.5 | mL |
| Water | 80% | v/v | 20 | mL |
| Sumatriptan Succinate | 25 | mg/mL | 625 | mg |
| Glycerin | 10% | v/v | 2.5 | mL |
| Potassium Sorbate | 1 | mg/mL | 25 | mg |

Formulations 6 and 6a were used as the basic formula to which a sweetener (saccharin sodium added at a concentration of 0.1 percent (w/v)), a bitter mask (added at a concentration of 0.06 percent (v/v) and commercially available from Firmenich S.A. of Geneva Switzerland), and finally cherry flavor (added at a concentration 0.06 percent (v/v) and commercially available from Firmenich S.A.) were added. The compositions of these formulations are provided below.

Formulation 6 with Sodium Saccharin

| | | Amount | |
|---|---|---|---|
| Ingredient | Amount | Per 25 mL | |
| Sorbitol | 20% v/v | 5 | mL |
| Water | 80% v/v | 20 | mL |
| Sumatriptan Succinate | 25 mg/mL | 625 | mg |
| PVP K25 | 44 mg/mL | 1.1 | g |
| Potassium Sorbate | 1 mg/mL | 25 | mg |
| Sodium Saccharin | 0.1% w/v | 25 | mg |

Formulation 6 with Sodium Saccharin and Bitter Mask

| | | Amount | |
|---|---|---|---|
| Ingredient | Amount | Per 25 mL | |
| Sorbitol | 20% v/v | 5 | mL |
| Water | 80% v/v | 20 | mL |
| Sumatriptan Succinate | 25 mg/mL | 625 | mg |
| PVP K25 | 44 mg/mL | 1.1 | g |
| Potassium Sorbate | 1 mg/mL | 25 | mg |
| Sodium Saccharin | 0.1% w/v | 25 | mg |
| Bitter Mask | 0.06% v/v | 16 | μL |

Formulation 6 with Sodium Saccharin, Bitter Mask, and Cherry Flavor

| | | Amount | |
|---|---|---|---|
| Ingredient | Amount | Per 25 mL | |
| Sorbitol | 20% v/v | 5 | mL |
| Water | 80% v/v | 20 | mL |
| Sumatriptan Succinate | 25 mg/mL | 625 | mg |
| PVP K25 | 44 mg/mL | 1.1 | g |
| Potassium Sorbate | 1 mg/mL | 25 | mg |
| Sodium Saccharin | 0.1% w/v | 25 | mg |
| Bitter Mask | 0.06% v/v | 16 | μL |
| Cherry Flavor | 0.06% v/v | 16 | μL |

Formulation 6a with Sodium Saccharin

| | | Amount | |
|---|---|---|---|
| Ingredient | Amount | Per 25 mL | |
| Sorbitol | 10% v/v | 2.5 | mL |
| Water | 80% v/v | 20 | mL |
| Sumatriptan Succinate | 25 mg/mL | 625 | mg |
| Glycerin | 10% v/v | 2.5 | mL |
| Potassium Sorbate | 1 mg/mL | 25 | mg |
| Sodium Saccharin | 0.1% w/v | 25 | mg |

Formulation 6a with Sodium Saccharin and Bitter Mask

| | | Amount | |
|---|---|---|---|
| Ingredient | Amount | Per 25 mL | |
| Sorbitol | 10% v/v | 2.5 | mL |
| Water | 80% v/v | 20 | mL |
| Sumatriptan Succinate | 25 mg/mL | 625 | mg |
| Glycerin | 10% v/v | 2.5 | mL |
| Potassium Sorbate | 1 mg/mL | 25 | mg |
| Sodium Saccharin | 0.1% w/v | 25 | mg |
| Bitter Mask | 0.06% v/v | 16 | μL |

Formulation 6a with Sodium Saccharin,
Bitter Mask, and Cherry Flavor

| | | Amount | |
|---|---|---|---|
| Ingredient | Amount | Per 25 mL | |
| Sorbitol | 10% v/v | 2.5 | mL |
| Water | 80% v/v | 20 | mL |
| Sumatriptan Succinate | 25 mg/mL | 625 | mg |
| Glycerin | 10% v/v | 2.5 | mL |
| Potassium Sorbate | 1 mg/mL | 25 | mg |
| Sodium Saccharin | 0.1% w/v | 25 | mg |
| Bitter Mask | 0.06% v/v | 16 | μL |
| Cherry Flavor | 0.06% v/v | 16 | μL |

These variations were tested at 50° C. for 504 hours. The data collected after this time is shown below.

| | Percent Degradation After |
|---|---|
| | 504 Hours at 50° C. |
| Formulation 6 | 0.699 |
| With Saccharin | 0.384 |
| With Saccharin and Bitter Mask | 0.12 |
| Formulation 6a | 0.44 |
| With Saccharin | 0.377 |
| With Saccharin and Bitter Mask | 0.089 |

After this time, all variations of Formulation 6 had a yellow color, including their controls, which had slightly less light yellow color change. Formulation 6a, however, had much less yellow color, and the controls did not have a color change. Analysis of these formulations demonstrated that Formulation 6a had better stability compared to Formulation 6. Surprisingly and unexpectedly, the stability of both Formulation 6 and 6a were increased upon the addition of saccharin, which was further increased when both saccharin and a bitter mask or bitterness-reducing agent were added.

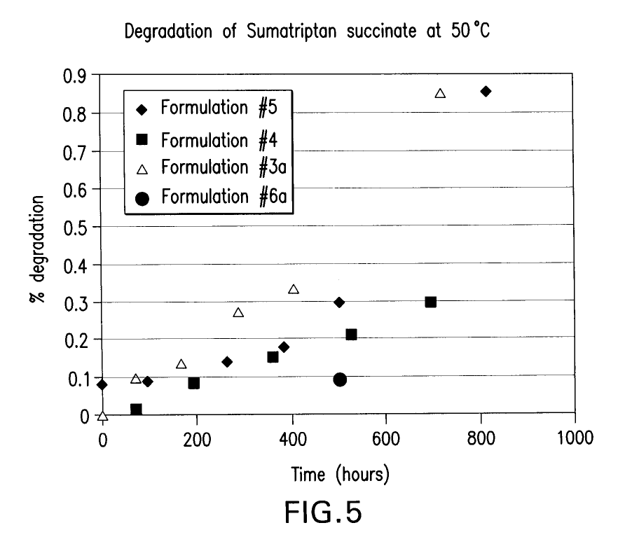

Finally, comparing the degradation of Formulation 6a (including saccharin, and bitterness-reducing agent) with the previous Formulations described demonstrates that Formulation 6a had the greatest stability. FIG. 5 illustrates the stability for Formulations 3a, 4, 5, and 6a.

Yet, even though Formulations 6a included a sweetening agent, bitterness-reducing agent and cherry flavor to minimize the bitterness of sumatriptan, the bitter taste of sumatriptan could be further masked. Therefore, a dark chocolate flavor was substituted for the cherry flavor. This change proved to be more effective in masking the bitter taste. Formulation 7 given below was found to effectively mask the bitter taste of sumatriptan succinate in solution at 20 mg/mL. Furthermore, the stability of this formulation was analyzed after two months at 40° C. and found to have approximately 0.8 percent degradation.

Formulation 7

| | | Amount Per |
|---|---|---|
| Ingredient | Amount | 5 mL dose |
| Water | 59.5% v/v | |
| Sumatriptan Succinate | 2.8% w/v | 141 mg |
| | | (100 mg base) |
| Glycerin | 20% v/v | |
| Sorbitol | 20% v/v | |
| Saccharin | 1% w/v | 50 mg |
| (or Sucralose) | (0.4% w/v) | (20 mg) |
| Potassium Sorbate | 0.1% w/v | 5 mg |
| Dark Chocolate Flavor | 0.16% v/v | |
| Brown Dye | 0.16% v/v | |
| Bitter Mask | 0.16% v/v | |

Additionally, an artificial milk chocolate and grapefruit flavor were also evaluated for their ability to mask the taste of sumatriptan in solution at 20 mg/mL. The artificial milk chocolate flavor using Formulation 7 with substituted artificial milk chocolate flavor was found to be more effective than the grapefruit flavor.

In order to determine the effect of potassium sorbate on Formulation 7, a comparison was made between the degradation of sumatriptan with or without potassium sorbate in the formulation. Additionally, the effects of the bitter mask, brown dye, artificial chocolate flavor, and sucralose were evaluated by comparing the stability of sumatriptan with and without these excipients. The following table presents the various combinations which were made for stability studies at 40° C.

TABLE 23

| | | | | | | | | |
|---|---|---|---|---|---|---|---|---|
| | | | | | | Chocolate | Brown | Bitter |
| Sumatriptan | Glycerin | Sorbitol | Saccharin | Sucralose | K+ sorbate | Flavor | Color | Mask |
| (mg/mL) | (% v/v) | (% v/v) | (% w/v) | (% w/v) | (% w/v) | (% v/v) | (% v/v) | (% v/v) |

Formulation 7a

| | | | | | | |
|---|---|---|---|---|---|---|
| 20 | 20 | 20 | 1 | 0.16 | 0.16 | 0.16 |

Formulation 7b

| | | | | | | |
|---|---|---|---|---|---|---|
| 20 | 20 | 20 | 0.4 | 0.16 | 0.16 | 0.16 |

Formulation 7c

| | | | | | | | |
|---|---|---|---|---|---|---|---|
| 20 | 20 | 20 | 1 | 0.1 | 0.16 | 0.16 | 0.16 |
| 20 | 20 | 20 | 1 | 0.1 | 0.16 | 0.16 | |
| 20 | 20 | 20 | 1 | 0.1 | 0.16 | | 0.16 |
| 20 | 20 | 20 | 1 | 0.1 | 0.16 | | |
| 20 | 20 | 20 | 1 | 0.1 | | 0.16 | 0.16 |
| 20 | 20 | 20 | 1 | 0.1 | | 0.16 | |
| 20 | 20 | 20 | 1 | 0.1 | | | 0.16 |
| 20 | 20 | 20 | 1 | 0.1 | | | |

Formulation 7d

| | | | | | | | |
|---|---|---|---|---|---|---|---|
| 20 | 20 | 20 | 0.4 | 0.1 | 0.16 | 0.16 | 0.16 |
| 20 | 20 | 20 | 0.4 | 0.1 | 0.16 | 0.16 | |
| 20 | 20 | 20 | 0.4 | 0.1 | 0.16 | | 0.16 |
| 20 | 20 | 20 | 0.4 | 0.1 | 0.16 | | |
| 20 | 20 | 20 | 0.4 | 0.1 | | 0.16 | 0.16 |
| 20 | 20 | 20 | 0.4 | 0.1 | | 0.16 | |
| 20 | 20 | 20 | 0.4 | 0.1 | | | 0.16 |
| 20 | 20 | 20 | 0.4 | 0.1 | | | |

In addition to the variations listed in Table 23, variations of Formulations 7c and 7d were created without adding potassium sorbate for comparison with the stability of the samples in Table 23. A formulation base of 20 mg/mL sumatriptan succinate, 20 percent (v/v) glycerin, 20 percent (v/v) sorbitol, 0.16 percent (v/v) brown dye, 0.16 percent (v/v) chocolate flavor, and 0.16 percent (v/v) bitterness-reducing agent or bitter mask (commercially available from Firmenich S.A.) was created.

The following table summarizes the stability of these batches after two months at 40° C.

TABLE 24

| | Percent |
|---|---|
| Composition | Degradation |
| Base + 1% (w/v) Saccharin Sodium | 0.079 |
| Base + 0.4% (w/v) Sucralose | 0.048 |
| Base + 1% (w/v) Saccharin Sodium + | 0.069 |
| 0.1% (w/v) Potassium Sorbate | |
| Base + 0.4% (w/v) Saccharin Sodium + | 0.056 |
| 0.1% (w/v) Potassium Sorbate | |
| Base - Bitter Mask + 1% (w/v) Saccharin | 0.029 |
| Sodium | |
| Base - Bitter Mask + 1% (w/v) Saccharin | 0.042 |
| Sodium + 0.1% (w/v) Potassium Sorbate | |
| Base - Bitter Mask + 0.4% (w/v) | 0.16 |
| Saccharin Sodium + 0.1% (w/v) | |
| Potassium Sorbate | |

Based on the results presented in this table, it is clear that using sucralose to sweeten the formulation generated increased stability compared to saccharin sodium. However, when potassium sorbate is used with saccharin, the degradation is lower than when no preservative is added. Additionally, the stability of this formulation was better without the bitter mask. Therefore, further studies were performed to determine whether the bitterness-reducing or masking agent could be eliminated from this formulation.

### Example 4

### Oral Liquid Sumatriptan Compositions without Conventional Bitterness-Reducing Agent According to the Invention

### Example 4.1

### Flavor and Taste Optimization

To further improve the flavor and suppress the bitter taste of sumatriptan, batches were prepared with sucralose as a sweetener and different flavors (chocolate, grape, and peppermint). Peppermint flavor in combination with sucralose was found to be the most effective in suppressing the bitter taste of sumatriptan. The sucralose and peppermint flavor synergistically function to reduce the bitterness of the sumatriptan. Based on subsequent trials with different peppermint flavors, Bell's Natural and Artificial Peppermint Flavor 113.24785 was selected for optimal taste masking. The green color of the formulation (with D&C Green #5 and D&C Yellow #10) was chosen to match with the mint flavor. Sucralose USP/NF from Tate & Lyle Sucrose, Inc. and D&C Green #5 from Noveon Hilton Davis, Inc. were used. The composition is given in Table 25 below.

TABLE 25

| Ingredient | Amount | | Amount per 5 mL |
|---|---|---|---|
| Sumatriptan succinate | 1% | w/v | 70 mg |
| | | | (50 mg base) |
| Sorbitol solution | 26% | w/v | 1.3 g |
| Glycerin | 37.5% | w/v | 1.875 g |
| Potassium sorbate | 0.1% | w/v | 5 mg |
| Sucralose | 0.4% | w/v | 20 mg |
| D&C Green #5 | 0.001% | w/v | 0.05 mg |
| D&C Yellow #10 | 0.001% | w/v | 0.05 mg |
| Peppermint Flavor | 0.4% | v/v | 20 μL |
| Water | | | Q.S. |

The taste of the formulation was further evaluated with respect to two parameters: (1) whether the concentration of the drug could be increased further in the formulation while retaining a palatable formulation; and (2) whether removing sorbitol and/or glycerin would significantly impact the flavor of the formulation.

To evaluate the effect of higher concentrations of the drug, formulations were prepared at 10, 20, 30, 40, 50 and higher mg/mL sumatriptan as sumatriptan succinate using the formulation of Table 25. Solutions at concentrations of 50 mg/mL and higher demonstrated could not be prepared due to limited drug solubility. A group of 5 volunteers evaluated the taste of 20 mg/mL and 30 mg/mL drug concentrations with the above-noted level of sweetener, and also with double the level of sweetener (sucralose). In the opinion of the panel, the bitter taste of the drug was clearly perceived at 20 mg/mL and was increasingly more pronounced at higher concentrations. This taste was not overcome by increasing the concentration of the sweetener. The 10 mg/mL concentration was deemed the best tasting by all. Therefore, the drug concentration should be no greater than about up to or below 40 mg/mL of sumatriptan to prepare a palatable oral solution formulation.

The exclusion of sorbitol and/or glycerin, and the inclusion of propylene glycol instead of both sorbitol and glycerin was evaluated to determine whether these excipients would adversely impact the taste of the formulation. The following formulations were reviewed by a panel of 5 volunteers:
a) Sorbitol and glycerin
b) No sorbitol and glycerin
c) No sorbitol, no glycerin, and propylene glycol
d) No sorbitol, glycerin, and propylene glycol
e) No sorbitol and no glycerin
f) Sorbitol and no glycerin

The volunteers considered formulation “a” good. Formulations “b” and “c” were considered equally good, with no perceived bitterness, and the mint flavor was stronger than in formulation “a.” Formulation “d” was considered similar to formulation “c,” while formulations “e” and “f” were definitely bitter. These observations indicated that glycerin and/or propylene glycol should be included in the formulation to provide sweetness and/or to reduce the bitterness of the drug.

### Example 4.2

### Stability Studies of Compositions According to the Invention and the Imitrex® Injection Formulation

The stability of the present oral formulation was compared with that of the conventional Imitrex® injection formulation at 4° C., 25° C., and 40° C. for three months. The HPLC analysis of the stability samples was conducted using separate methods for Impurity A, Impurities C and D, and the assay. Stability data of the present oral formulation in HDPE bottle pack compared to the conventional Imitrex® injection formulation is presented below. Levels of each impurity are expressed as percentage (w/w) of sumatriptan, and RT is the retention time in minutes. While the degradation data presented in previous examples represented total amount of impurities as a percentage of total drug peak area, this and succeeding examples present individual impurities based on improved methods that were developed, as detailed in Table 27.

TABLE 26

| | | Suma- | K | Column for Impurity A | | | | Column for Impurities C and D | | | | | | | | | | |
|---|---|---|---|---|---|---|---|---|---|---|---|---|---|---|---|---|---|---|
| | | triptan | Sorbate | RT | RT | RT | RT 8.4 | RT | RT | RT | RT | RT | RT | RT | RT 27.9 | RT 28.1 | RT | RT |
| Temp | Time | assay** | assay** | 3.9 | 4.2 | 6.4 | (Imp A) | 13.1 | 18.3 | 20.3 | 21.0 | 22.6 | 22.7 | 23.0 | (Imp C) | (Imp D) | 29.0 | 30.5 |
| Peak # for this report: | | | | 1 | 2 | 3 | 4 | 5 | 6 | 7 | 8 | 9 | 10 | 11 | 12 | 13 | 14 | 15 |
| 4° C. | 1 M | 100.1 | 100.9 | 0.03 | 0.02 | * | * | * | * | 0.01 | * | 0.01 | * | * | * | 0.02 | * | * |
| | 2 M | 100.9 | 101.5 | 0.03 | 0.02 | * | 0.03 | * | * | * | * | 0.01 | * | * | 0.03 | * | * | * |
| | 3 M | 101.3 | 98.6 | * | * | * | 0.05 | * | * | * | * | 0.03 | 0.02 | * | 0.02 | 0.03 | * | * |
| 25° C. | 1 M | 99.4 | 100.2 | 0.02 | 0.02 | 0.02 | * | * | * | 0.02 | * | 0.01 | * | * | * | 0.02 | * | * |
| | 2 M | 101.2 | 101.6 | 0.02 | 0.02 | 0.03 | 0.02 | * | * | 0.02 | 0.02 | 0.01 | * | * | 0.04 | * | * | * |
| | 3 M | 100.9 | 97.8 | * | * | 0.01 | 0.05 | * | * | * | 0.03 | 0.02 | 0.02 | * | 0.02 | 0.03 | * | * |
| 40° C. | 1 M | 99.4 | 99.3 | 0.02 | 0.02 | 0.06 | * | 0.02 | * | 0.07 | 0.02 | 0.01 | * | 0.02 | * | 0.02 | 0.02 | * |
| | 2 M | 99.6 | 98.4 | 0.04 | 0.03 | 0.12 | 0.01 | 0.02 | 0.04 | 0.11 | 0.03 | * | * | 0.02 | 0.04 | * | 0.04 | * |
| | 3 M | 100.4 | 94.5 | * | * | 0.09 | 0.04 | 0.03 | * | 0.17 | 0.03 | 0.03 | 0.02 | * | 0.02 | 0.03 | 0.05 | * |

Imitrex ® Injection 12 mg/mL

| | | | | | | | | | | | | | | | | | | |
|---|---|---|---|---|---|---|---|---|---|---|---|---|---|---|---|---|---|---|
| 4° C. | 3 M | na | NA | * | * | 0.22 | 1.57 | 0.04 | 0.04 | 0.13 | * | 0.13 | 0.09 | * | * | * | 0.12 | * |
| 25° C. | 3 M | na | NA | * | * | 0.04 | 1.64 | 0.01 | 0.03 | 0.05 | * | 0.11 | 0.09 | * | * | * | 0.12 | * |
| 40° C. | 3 M | na | NA | * | * | 0.07 | 2.16 | 0.05 | 0.06 | 0.07 | * | 0.05 | 0.07 | * | * | * | 0.15 | * |

*, not detected;
**, reported as % of label claim;
NA, not applicable;
na, not available

Information regarding the column, chromatographic conditions, and mobile phase gradient used in the testing are provided below in Table 27.

TABLE 27

| | | Chromatographic | |
|---|---|---|---|
| Method for | Column | conditions | Mobile phase gradient |
| Assay | SieLC, Primesep | Wavelength 285 nm, | A: 0.05% trifluoroacetic acid (TFA) in |
| | AB, 5 μm, 250 mm × 4.6 mm, | flow rate 1 mL/min, | Water |
| | Part No. AB- | inj vol 10 μL, temp | B: 0.2% TFA in 20/80 water/acetonitrile |
| | 46.250.0510 | 40° C. | (CAN) |
| | | | C: 0.2% TFA in CAN |
| | | | Gradient: 5% B and 95% A at 0 min to 50% |
| | | | each at 20 min, to 100% C at 21 min, hold |
| | | | until 30 min |
| Impurity A | Waters, | Wavelength 285 nm, | 10 M Ammonium Acetate Buffer: |
| | Spherisorb | flow rate 1 mL/min, | Methanol (9:1), isocratic for 15 min |
| | Silica, 5 μm, 250 mm × 4.6 mm, | inj vol 50 μL, temp | |
| | Part No. 8376 | 40° C. | |
| Impurities C | SieLC, Primesep | Wavelength 285 nm, | A: 0.05% TFA in Water |
| and D | P, 5 μm, 250 mm × 4.6 mm, | flow rate 1 mL/min, | B: 0.2% TFA in 20/20/80 |
| | Part | inj vol 50 μL, temp | water/MeOH/CAN |
| | No. P- | 40° C. | C: 0.2% TFA in CAN |
| | 46.250.0510 | | Gradient: 5% B and 95% A at 0 min to 100% |
| | | | B at 40 min, keep until 50 min, to 100% C at |
| | | | 51 min, hold until 60 min |

Review of the impurity data indicated the presence of one unknown impurity at peak #7 that increased with an increase in temperature as well as incubation time. The level of this impurity in the 40° C./three month sample was 0.17%, which is very close to the regulatory qualification limit of 0.20%. Therefore, further studies were conducted to investigate the cause of formation of this impurity.

The conventional Imitrex® injection formulation also showed the presence of the same impurity at a significant level. However, the levels of the impurity at peak #7, as well as that of the impurities at peak numbers 3 and 9 were surprisingly higher at 4° C. than at 40° C. Furthermore, impurity A at peak #4 was observed at a significantly high level at all temperatures.

### Example 5

### Characterization of Unknown Impurity

To better identify the unknown impurity, Liquid Chromatography-Mass Spectroscopy (LC-MS) of the formulation was carried out. HPLC conditions were the same as that for impurity C and D analysis, and the product peaks were injected into an electrospray ionization mass spectrometer. From the mass spectra, the molecular weight of the unknown compound was inferred to be 312. This molecular weight represented an oxygen atom addition to the sumatriptan molecule, indicating it as an oxidation product.

To confirm whether oxidation of sumatriptan would lead to the formation of this impurity, sumatriptan succinate was incubated with 25% H₂O₂ for 2 hours and 24 hours followed by LC-MS analysis. The HPLC chromatogram showed an increase in peak area at the same retention time, and mass spectroscopy confirmed that it had the same molecular weight, thus confirming it as an oxidation product of sumatriptan.

To confirm the presence of the same impurity in the brand name product, the conventional Imitrex® injection formulation was analyzed by LC-MS. Observation of the same retention time and same molecular weight confirmed that this impurity was also present in the conventional Imitrex® product.

To test the presence of this impurity in sumatriptan succinate procured from different vendors, solid sumatriptan in powder form as well as sumatriptan dissolved in water were analyzed by HPLC. This oxidation impurity was not present in the solid sumatriptan but formed upon incubation of the aqueous solution at 50° C. for two weeks. This indicated the potential for formation of the oxidation impurity in the aqueous drug solutions even if it was not present in the sumatriptan.

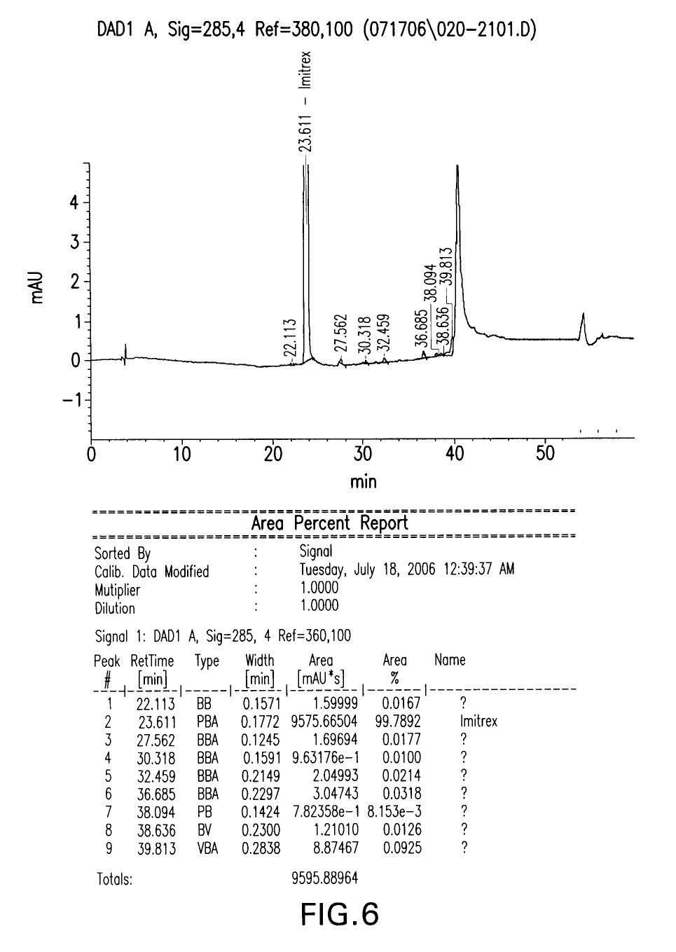

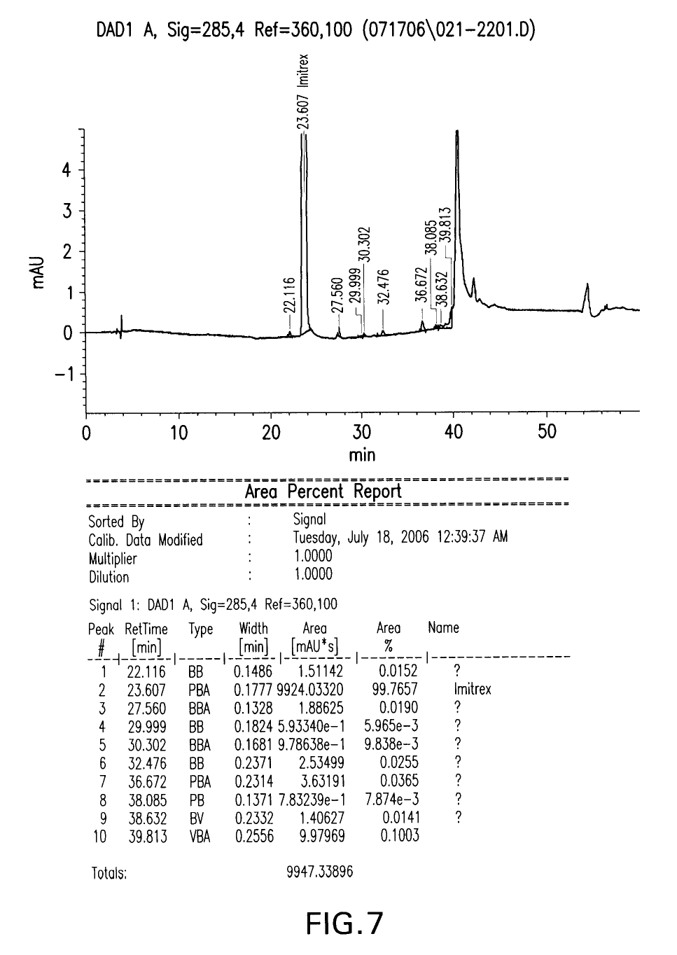

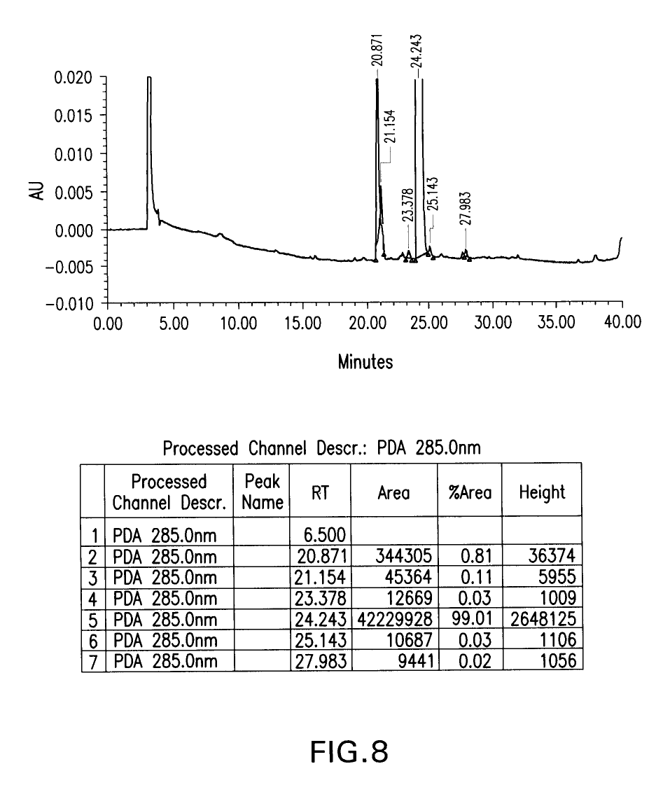

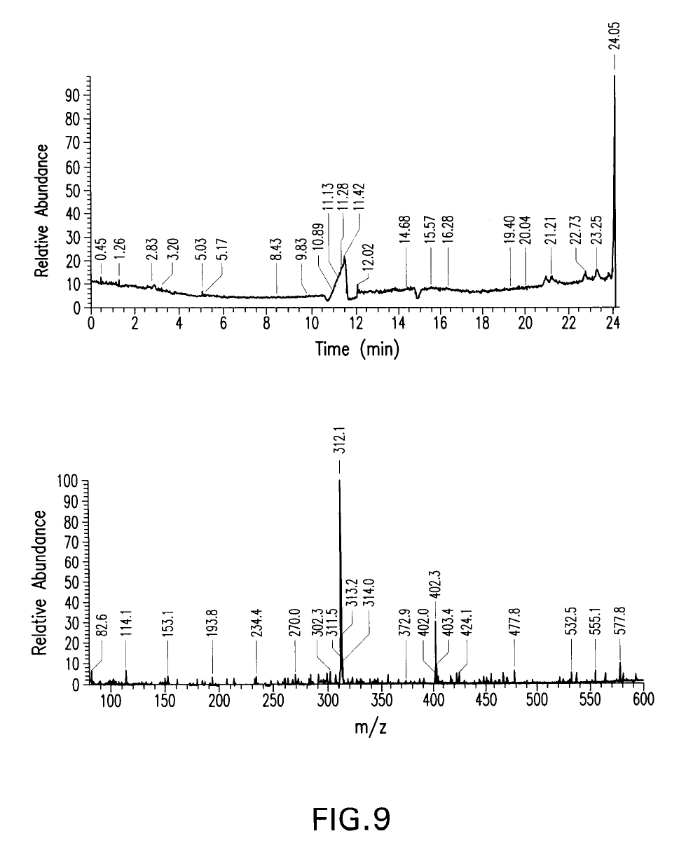

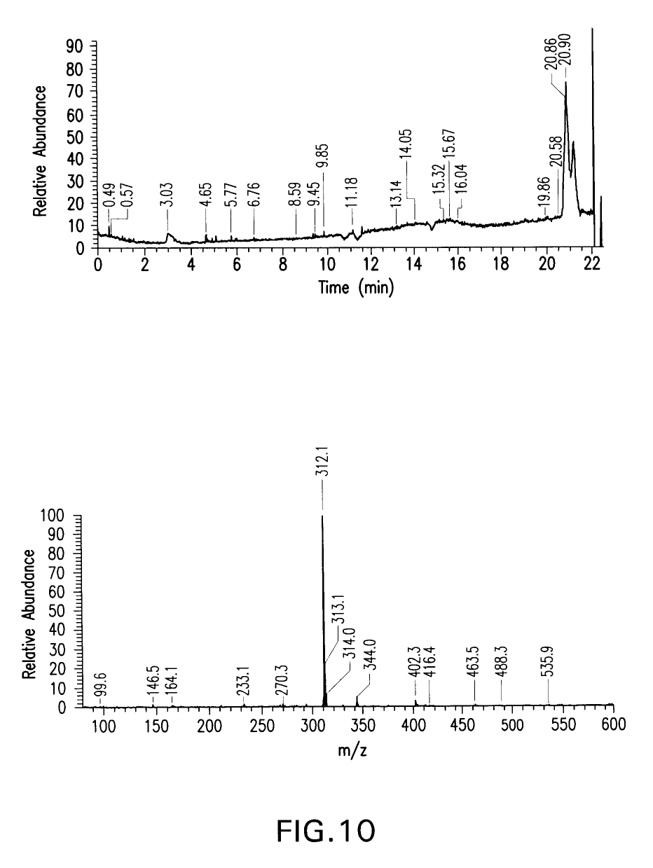

HPLC chromatograms and mass spectra for this impurity in the formulation, sumatriptan, and Imitrex® injection samples are presented here in FIGS. 6-10. FIGS. 6 and 7 are the chromatograms showing absence of this impurity in solid drug at both 4° C. and 50° C., respectively. FIG. 8 shows its formation in aqueous solutions upon incubation for two weeks at 50° C. The impurity had a retention time of 20.871 minutes.

Thus, oxidation was identified as a mode of drug degradation in the aqueous formulation. The impurity thus formed was different from any of the known impurities in USP, Ph. Eur., and vendor literature. If this impurity is not controlled within the regulatory qualification limit of 0.20%, it would warrant toxicological evaluation. Hence, it is of critical importance in the development of oral liquid formulations of sumatriptan to identify and use measures to control the formation and growth of this impurity in the formulation.

### Example 6

### Factors Affecting Drug Oxidation

To identify whether the oxidation of drug in the formulation was due to the oxygen dissolved in the aqueous phase, the following trial was conducted: elimination of sorbitol and/or glycerin with or without addition of propylene glycol.

### Example 6.1

### Elimination of Sorbitol and/or Glycerin

To evaluate whether the density and viscosity adding ingredients of the formulation have a role in enhancing oxidative degradation of the drug, formulations were prepared with and without glycerin and sorbitol. In addition, in some formulations, glycerin was replaced or combined with propylene glycol to evaluate whether the use of a relatively hydrophobic ingredient would prevent oxidative drug degradation. These formulations were packed in 1 oz HDPE bottle packs at 30 mL/bottle fill volume and kept at 40° C. for one month and two months. The samples were then analyzed by HPLC using the method for impurities C and D.

The results, summarized in Table 28 (one month) and Table 29 (two months), indicate that the presence of sorbitol made a significant contribution to drug degradation. Elimination of sorbitol prevented the growth of the impurity at peak #3. The reason for greater influence of sorbitol than glycerin may be due to its higher density (1.3 for sorbitol volumes, and 1.25 for glycerin) and the presence of double the number of hydroxyl groups in the molecule, which play a role in hydrogen bonding that helps dissolve and keep the dissolved oxygen in water. Thus, the presence of excipients with a hydrogen bonding capacity for O₂ and increase in viscosity/density of the formulation could play a role in increasing oxidative degradation of the drug. In addition, without sorbitol, including propylene glycol helped to minimize the impurity at peak #7. Levels of each impurity are expressed as percentage (w/w) of sumatriptan, and RT is the retention time in minutes.

TABLE 28

| | RT | RT | RT | RT | RT |
|---|---|---|---|---|---|
| Excipient | 17.46 | 19.42 | 19.82 | 27.23 | 28.10 |
| Peak # | 6 | 7 | 8 | 13 | 14 |

With sorbitol

| | | | | | |
|---|---|---|---|---|---|
| Glycerin | 0.020 | 0.060 | 0.013 | 0.040 | 0.021 |
| With PG | 0.029 | 0.080 | 0.024 | 0.037 | 0.025 |
| With PG + Glycerin | 0.019 | 0.062 | 0.015 | 0.040 | 0.020 |

Without sorbitol

| | | | | | |
|---|---|---|---|---|---|
| Glycerin | 0.012 | 0.027 | 0 | 0.040 | 0.017 |
| With PG | 0 | 0.019 | 0.007 | 0.040 | 0.021 |
| With PG + Glycerin | 0.012 | 0.014 | 0 | 0.039 | 0.0170 |

TABLE 29

| | | | | | | Method for | |
|---|---|---|---|---|---|---|---|
| | Method for Impurities C & D | | | | | Impurity A | |
| | RT | RT | RT | RT | RT | RT | RT |
| Excipient | 17.46 | 19.42 | 19.82 | 27.23 | 28.10 | 6.73 | 8.96 |
| Peak # | 6 | 7 | 8 | 13 | 14 | 3 | 4 |

With sorbitol

| | | | | | | | |
|---|---|---|---|---|---|---|---|
| Glycerin | 0.046 | 0.107 | 0.013 | 0 | 0.042 | 0 | 0 |
| With PG | 0.067 | 0.146 | 0.019 | 0.040 | 0.041 | 0.132 | 0 |
| With PG + | 0.048 | 0.110 | 0.025 | 0.042 | 0.039 | 0.096 | 0 |
| Glycerin | | | | | | | |

Without sorbitol

| | | | | | | | |
|---|---|---|---|---|---|---|---|
| Glycerin | 0.021 | 0.055 | 0.014 | 0.044 | 0.048 | 0.043 | 0.021 |
| With PG | 0.012 | 0.040 | 0.011 | 0.046 | 0.048 | 0.033 | 0.020 |
| With PG + | 0.019 | 0.052 | 0.013 | 0.042 | 0.047 | 0.042 | 0 |
| Glycerin | | | | | | | |

The stability of sample batches made without sorbitol were analyzed for two months at 4° C. and 25° C. to confirm that the formulation was stable at both room temperature and refrigerated conditions. This data is presented in Table 30. Levels of each impurity are expressed as percentage (w/w) of sumatriptan, and RT is the retention time in minutes.

TABLE 30

| | | | | | | | Method for | |
|---|---|---|---|---|---|---|---|---|
| | | Method for Impurities C & D | | | | | Impurity A | |
| | | RT | RT | RT | RT | RT | | RT |
| Excipient | Temp | 17.46 | 19.42 | 19.82 | 27.23 | 28.10 | RT 6.73 | 8.96 |
| Peak # | | 6 | 7 | 8 | 13 | 14 | 3 | 4 |
| Glycerin | 40° C. | 0.021 | 0.055 | 0.014 | 0.044 | 0.048 | 0.043 | 0.021 |
| | 25° C. | 0 | 0.045 | 0 | 0.040 | 0 | 0.012 | 0 |
| | 4° C. | 0 | 0 | 0 | 0.039 | 0 | 0 | 0 |
| With PG | 40° C. | 0.012 | 0.040 | 0.011 | 0.046 | 0.048 | 0.033 | 0.020 |
| | 25° C. | 0 | 0.016 | 0.011 | 0.039 | 0 | 0.013 | 0 |
| | 4° C. | 0 | 0.014 | 0 | 0.039 | 0 | 0.012 | 0 |
| With PG + Glycerin | 40° C. | 0.019 | 0.052 | 0.013 | 0.042 | 0.047 | 0.042 | 0 |
| | 25° C. | 0 | 0.046 | 0 | 0.039 | 0 | 0.011 | 0 |
| | 4° C. | 0 | 0.011 | 0 | 0.040 | 0 | 0.011 | 0 |

This data indicated that the formulation remained stable at all temperature conditions. It further demonstrated that inclusion of glycerin led to a temperature dependent increase in the impurity at peak #7, while inclusion of propylene glycol alone stabilized the formulation at all temperature conditions. Therefore, a formulation with propylene glycol was considered advantageous, while a formulation without propylene glycol and including glycerin was also suitable.

To identify the role of formulation variables in influencing formation of the oxidation impurity, the following trials were conducted:

1. Formulation at different pH

2. Different levels of excipients

3. Incorporation of antioxidants

### Example 6.2

### Formulation at Different pH

Sumatriptan succinate has a self-buffering capacity due to it being a succinic acid salt. Sumatriptan succinate has pKa values of 4.21 and 5.67 from succinic acid, 9.63 from the tertiary amine, and greater than 12 from the sulfonamide group. The pH of sumatriptan succinate solutions is between 4.5-5, while that of placebo is greater than 6, confirming the role of the sumatriptan in modulating the pH of the solution. The pH range of the conventional Imitrex® injection formulation, 4.2-5.3, is similar to the preferred pH range of the inventive formulations.

To investigate whether buffering the solution to a pH level higher or lower would affect formation of the oxidation impurity, formulations were prepared and adjusted to pH 4.5 and 7.5. These formulations were kept on stability studies at 50° C. for two weeks. Analysis of stability samples by HPLC method for impurities C and D indicated that higher pH accelerated degradation. Buffered formulations at pH 4.5 showed similar levels of impurity at peak #7 than unbuffered formulation. The latter is preferable since it minimizes excipients without any adverse effect on buffering and stability of the formulation. Therefore, self-buffered sumatriptan succinate formulations were deemed optimum, and the formulations of the invention therefore preferably may be substantially or entirely buffer-free.

Results are provided below. Levels of each impurity are expressed as percentage (w/w) of sumatriptan, and RT is the retention time in minutes.

TABLE 31

| | Temp | RT | RT | RT | RT | RT | RT | RT | RT | RT | RT |
|---|---|---|---|---|---|---|---|---|---|---|---|
| pH | (° C.) | 13.109 | 17.400 | 17.933 | 19.885 | 20.255 | 22.106 | 22.482 | 24.495 | 27.547 | 28.472 |
| Peak # | | 5 | — | 6 | 7 | 8 | 9 | 10 | — | 12 | 14 |
| 4.5 | 4 | 0 | 0 | 0 | 0.032 | 0 | 0.009 | 0 | 0 | 0.043 | 0 |
| | 50 | 0.025 | 0 | 0.038 | 0.133 | 0.045 | 0 | 0 | 0 | 0.038 | 0.034 |
| 7.5 | 4 | 0 | 0 | 0 | 0.033 | 0 | 0.015 | 0 | 0.121 | 0.036 | 0 |
| | 50 | 0.160 | 0.066 | 0.049 | 0.757 | 0.043 | 0 | 0.075 | 0.484 | 0.053 | 0.137 |
| Retention time | | | 13.215 | 15.122 | 19.712 | 20.093 | 22.076 | 22.464 | 27.736 | | 28.678 |
| a | 50 | | 0.039 | 0.011 | 0.116 | 0.034 | 0.015 | 0.012 | 0.025 | | 0.117 |
| (no buffer) | | | | | | | | | | | |

### Example 6.3

### Incorporation of Antioxidants

Use of antioxidants can potentially control the formation and/or increase in the level of the oxidation impurity. In the formulation of Table 25, however, only water soluble antioxidants were used. The following antioxidants were incorporated in the sumatriptan succinate formulation at recommended concentrations: 0.01 percent (w/v) ascorbic acid, 0.03 percent (w/v) a-tocopherol, 0.3 percent (w/v) sodium metabisulfite, 0.1 percent (w/v) fumaric acid, 0.1 percent (w/v) propyl gallate, and 0.07 percent (w/v) ethylene diamine tetraacetic acid (EDTA). Batches were kept at 40° C. for one month and two months, followed by HPLC analysis using the method for impurities C and D to identify the levels of oxidation impurity. Results of the analysis are presented in Tables 32 (one month) and 33 (two months). Levels of each impurity are expressed as percentage (w/w) of sumatriptan, and RT is the retention time in minutes.

TABLE 32

| | RT | RT | RT | RT |
|---|---|---|---|---|
| Antioxidant | 17.46 | 19.42 | 19.82 | 28.10 |
| Peak # | 6 | 7 | 8 | 14 |
| Without antioxidant | 0.011 | 0.027 | 0.008 | 0.011 |
| Ascorbic acid | 0.010 | 0.064 | 0.012 | 0 |
| α-Tocopherol | 0.010 | 0.029 | 0.009 | 0.012 |
| Na Metabisulfite | 0.270 | 0.088 | 0.564 | 0.063 |
| Fumaric acid | 0 | 0.027 | 0.009 | 0.007 |
| Propyl gallate | 0 | 0.012 | 0 | 0 |

TABLE 33

| | | | | | Method for | |
|---|---|---|---|---|---|---|
| | Method for Impurities C & D | | | | Impurity A | |
| | RT | RT | RT | RT | RT | RT |
| Antioxidant | 17.46 | 19.42 | 19.82 | 28.10 | 6.73 | 8.96 |
| Peak # | 6 | 7 | 8 | 14 | 3 | 4 |
| Without antioxidant | 0.046 | 0.107 | 0.013 | 0.042 | 0 | 0 |
| Ascorbic acid | 0.053 | 0.159 | 0.013 | 0.033 | 0.128 | 0.026 |
| α-Tocopherol | 0.033 | 0.089 | 0.017 | 0.041 | 0.075 | 0 |
| Na Metabisulfite | 0.588 | 0.199 | 0.776 | 0.033 | 0.250 | 0 |
| EDTA | 0.032 | 0.096 | 0.019 | 0.040 | 0.088 | 0 |
| Fumaric acid | 0.027 | 0.083 | 0.016 | 0.043 | 0.071 | 0 |
| Propyl gallate | 0 | 0.059 | 0 | 0.034 | 0.031 | 0.025 |

These experiments demonstrated that the oxidative degradation of the drug was primarily a factor of the oxygen dissolved in the aqueous media. This implies that nitrogen flushing of water to remove dissolved oxygen prior to formulation/packaging may help prevent the formation/growth of the oxidative impurity. In addition, the data reported above further indicated that the incorporation of propyl gallate as an antioxidant and/or minimizing or excluding sorbitol may also be helpful.

These results indicate that propyl gallate was the most effective in controlling oxidative degradation of the drug. The data indicated that propyl gallate significantly prevented the growth of the impurity at peak #7 as compared to the sample without any antioxidant, and better than any other antioxidant. The stability of sample batches made with propyl gallate for two months over 4° C. and 25° C. was evaluated to confirm that the formulation was stable at both room temperature and refrigerated conditions. This data is presented in Table 34.

TABLE 34

| | | | | | Column for | |
|---|---|---|---|---|---|---|
| | Column for Impurities C & D | | | | Impurity A | |
| | RT | RT | RT | RT | RT | RT |
| Temperature | 17.46 | 19.42 | 19.82 | 28.10 | 6.73 | 8.96 |
| Peak # | 6 | 7 | 8 | 14 | 3 | 4 |
| 40° C. | 0 | 0.059 | 0 | 0.034 | 0.031 | 0.025 |
| 25° C. | 0 | 0.043 | 0.030 | 0.042 | 0.018 | 0.026 |
| 4° C. | 0 | 0.032 | 0.015 | 0.032 | 0.021 | 0 |

This data indicates that the formulation remained stable at all temperature conditions. It further shows a temperature dependent increase in the impurity at peak #7. The level of the impurity for two months at 40° C. with the inclusion of propyl gallate (0.059 percent) is comparable to batches made without sorbitol (0.055 percent with glycerin and 0.052 percent without glycerin) and higher than that with the inclusion of propylene glycol alone (0.04 percent). Also, this formulation was made with 0.1 percent propyl gallate, while its maximum level of current use in an FDA approved drug product is only 0.02 percent (w/v). It is possible that propyl gallate may not be as effective at lower concentrations. Therefore, of all the antioxidants, a formulation with propyl gallate was considered to be particularly advantageous based on this preliminary testing.

### Example 7

### Open Bottle/Room Temperature Study

Sumatriptan succinate did not show appreciable oxidative degradation in the formulation at 4° C. and 25° C. storage conditions for three months. In order to confirm that the formulation would remain stable under the patient-handling conditions of room temperature storage, a study was initiated to keep the formulation in an open bottle for a one month and 10 week period followed by complete analysis. Results of this study are presented in Table 35 (one month) and 36 (10 weeks). Levels of each impurity are expressed as percentage (w/w) of sumatriptan, and RT is the retention time in minutes.

TABLE 35

| | | | | Column for | | | | | |
|---|---|---|---|---|---|---|---|---|---|
| | | Sumatriptan | | Impurity A | | Column for Impurities C and D | | | |
| Temp | Time | assay** | K Sorbate assay** | RT 6.24 | RT 8.30 | RT 17.46 | RT 19.42 | RT 19.82 | RT 28.10 |
| Impurity # | | | | 3 | 4 | 6 | 7 | 8 | 14 |
| 25° C. | 1 M | 107.0 | 107.5 | 0 | 0 | 0 | 0 | 0 | 0 |

**reported as % of label claim

TABLE 36

| | | | | Method for | | Method for Impurities | | | |
|---|---|---|---|---|---|---|---|---|---|
| | | Sumatriptan | | Impurity A | | C and D | | | |
| Temp | Time (weeks) | assay* | K Sorbate assay* | RT 6.73 | RT 8.96 | RT 17.46 | RT 19.42 | RT 19.82 | RT 28.10 |
| Impurity # | | | | 3 | 4 | 6 | 7 | 8 | 14 |
| 25° C. | 10 | 134.6 | 134.6 | 0.012 | 0.028 | 0 | 0.016 | 0.008 | 0.030 |

Higher assay values for both sumatriptan and potassium sorbate observed in these samples could be due to evaporation of the water during storage. The impurities, however, did not show any increase over this period of time. These results indicate that the formulation remained chemically stable even in an open bottle at room temperature for over 2 months. Thus, the formulation is stable under even the harshest of expected conditions under patient use, and it is believed that the carrier itself is critical to stability of the oral liquid sumatriptan formulations of the invention.

Although preferred embodiments of the invention have been described in the foregoing description, it will be understood that the invention is not limited to the specific embodiments disclosed herein but is capable of numerous modifications by one of ordinary skill in the art. It will be understood that the materials used and the chemical or pharmaceutical details may be slightly different or modified from the descriptions herein without departing from the methods and compositions disclosed and taught by the present invention.

What is claimed is:

1. An oral liquid composition comprising sumatriptan, or a pharmaceutically acceptable salt or metabolite thereof, and a pharmaceutically acceptable carrier that provides a liquid portion of the composition and is selected to minimize formation of one or more oxidation impurities, wherein the composition is substantially free of at least one of the oxidation impurities after storage for at least about one month at 40° C.

2. The composition of claim 1, wherein the carrier comprises one or more of a solubilizing agent, thickening agent, sweetening agent, flavoring agent, colorant agent, preservative agent, or antioxidant component.

3. The composition of claim 2, wherein the carrier comprises glycerin, the sweetening agent is present and comprises sorbitol or sucralose, the preservative agent is present and comprises a sorbate-containing component, the flavoring agent is present and provides a mint flavor, or the antioxidant component is present and comprises a gallate-containing component, or any combination thereof.

4. The composition of claim 3, wherein the sumatriptan, or salt or metabolite thereof, is present in an amount of about 0.5 percent to 5 percent (w/v) of the composition, and the carrier comprises glycerin present in an amount of about 5 percent to 60 percent (v/v), sorbitol solution present up to an amount of about 60 percent (w/v), sucralose present in an amount of about 0.1 percent to 1.5 percent (w/v), colorant agent present in an amount of about 0.0001 percent to 0.05 percent (w/v), potassium sorbate present in an amount of about 0.01 percent to 0.5 percent (w/v), peppermint flavor present in an amount of about 0.01 percent to 1 percent (v/v), and propyl gallate present in an amount of about 0.01 to 0.25 percent (w/v).

5. The composition of claim 1, wherein the oxidation impurity is present in an amount of less than about 0.5 percent (w/w) of the sumatriptan, or salt or metabolite thereof.

6. A method of preventing, treating, or managing migraine attacks in a mammal which comprises administering to the mammal a pharmaceutically effective amount of the composition of claim 1.

7. The method of claim 6, wherein the composition is administered one to four times a day.

8. The method of claim 7, wherein the total daily dose of sumatriptan is from about 25 mg to 200 mg.

9. An oral liquid composition comprising sumatriptan, or a pharmaceutically acceptable salt or metabolite thereof, and a pharmaceutically acceptable carrier that includes a liquid portion of the composition and comprises a sweetening agent and a flavoring agent that provides a mint flavor, the sweetening agent and flavoring agent included in amounts sufficient to collectively minimize or mask the bitterness of the sumatriptan.

10. The composition of claim 9, wherein the mint flavor comprises spearmint, peppermint, or a combination thereof.

11. The composition of claim 9, wherein the sweetening agent comprises sucralose.

12. The composition of claim 11, wherein the sucralose is present in an amount of about 0.1 percent to 0.6 percent (w/v) of the composition, and the flavoring agent is present in an amount of about 0.01 percent to 1 percent (v/v).

13. The composition of claim 9, wherein the sumatriptan, or salt or metabolite thereof, is present in an amount of less than or equal to about 40 mg/mL of sumatriptan base, of the composition.

14. The composition of claim 9, wherein the carrier further comprises one or more of a wetting agent, surfactant component, stabilizing agent, solubilizing agent, thickening agent, perfuming agent, colorant agent, preservative agent, or buffering agent.

15. The composition of claim 14, wherein the carrier comprises glycerin and sorbitol, the sweetening agent is present and comprises a sucralose, acesulfame, aspartame or saccharin-containing component, the preservative agent is present and comprises a sorbate-containing component, and the buffering agent is present and comprises an acetate, phosphate, or citrate-containing buffer.

16. A method of preparing an oral liquid sumatriptan composition which comprises:
dissolving an amount of sumatriptan, or a pharmaceutically acceptable salt or metabolite thereof, into at least one liquid adjuvant to form a liquid sumatriptan solution; and
combining the liquid sumatriptan solution with a sweetening agent and a flavoring agent that provides a mint flavor to form the oral liquid sumatriptan composition.

17. The method of claim 16, further comprising adding a bitterness-reducing agent.

18. The method of claim 16, wherein the at least one liquid adjuvant comprises water, a buffering agent, or both.

19. The method of claim 16, wherein the oral liquid sumatriptan composition is a clear solution.

20. An oral liquid composition comprising sumatriptan, or a pharmaceutically acceptable salt or metabolite thereof, and a pharmaceutically acceptable carrier that includes a liquid portion of the composition and comprises a bitterness-reducing agent and a flavoring agent.

21. The composition of claim 20, wherein the bitterness-reducing agent is present in an amount of about 0.01 percent to 1 percent (v/v) of the composition, the flavoring agent is present in an amount of about 0.01 percent to 1 percent (v/v) of the composition, or both.

22. The composition of claim 20, wherein the bitterness-reducing agent comprises monoammonium glycyrrhizinate or menthol, or a combination thereof.

23. The composition of claim 20, wherein the flavoring agent provides a flavor of chocolate, peppermint, cinnamon, coffee, grape, grapefruit, vanilla, orange, lemon, lime, mango, strawberry, pineapple, cherry, or a combination thereof.

24. A substantially stable oral liquid sumatriptan composition comprising the composition of claim 20, wherein one or more of the bitterness-reducing agent, the flavoring agent, and a sweetening agent are each present in an amount sufficient to reduce the bitter taste of sumatriptan.

25. The composition of claim 24, wherein the sweetening agent comprises sucralose and the flavoring agent provides a mint flavor; the sweetening agent comprises acesulfame and the flavoring agent provides an artificial chocolate flavor; or the sweetening agent comprises saccharin and the flavoring agent provides a grape flavor.

26. The composition of claim 20, wherein the composition is at least substantially stable and comprises sumatriptan succinate.

27. The composition of claim 20, wherein degradation of sumatriptan, or the salt or metabolite thereof, over a period of no more than about one month is no more than about 2 percent (w/w) at 50° C.
